(19) United States
(12) Patent Application Publication
(10) Pub. No.: US 2009/0111757 A1
(43) Pub. Date: Apr. 30, 2009

(54) HCV PROTEASE INHIBITORS

(75) Inventors: Chu-Chung Lin, Taipei City (TW); Kuang-Yuan Lee, Hsinchu City (TW); Chen-Fu Liu, Taipei City (TW); Pin Lo, Taipei County (TW); Yo-Chin Liu, Taipei County (TW); Yueh-chiang Han, Taipei County (TW); Chi-Hsin Richard King, Holladay, UT (US)

Correspondence Address: OCCHIUTI ROHLICEK & TSAO, LLP, 10 FAWCETT STREET, CAMBRIDGE, MA, 02138, US

(73) Assignee: TaiGen Biotechnology Co., Ltd., Taipei (TW)

(21) Appl. No.: 12/254,439

(22) Filed: Oct. 20, 2008

Related U.S. Application Data
(60) Provisional application No. 60/982,604, filed on Oct. 25, 2007.

(51) Int. Cl. A61K 38/07; C07K 5/103

(52) U.S. Cl. 514/18; 530/330

(57) ABSTRACT

Compounds of formula (I):

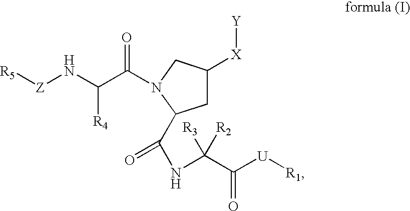

in which R₁, R₂, R₃ R₄, and R₅, U, X, Y, and Z are as defined herein. Also disclosed is use of these compounds, alone or in combination with other active agents, to treat hepatitis C virus infection.

### CROSS REFERENCE

This application claims priority to U.S. Provisional Application Ser. No. 60/982,604, filed Oct. 25, 2007, the contents of which are incorporated herein by reference.

### BACKGROUND

Hepatitis C virus (HCV), a (+)-sense single-stranded RNA virus, is the major causative agent for most cases of non-A, non-B hepatitis. It has been implicated in liver cirrhosis and hepatocellular carcinoma. Infection by HCV is a compelling human health problem. See, e.g., WO 05/007681; WO 89/04669; EP 381216; Alberti et al., J. Hepatology, 31 (Suppl. 1), 17-24 (1999); Alter, J. Hepatology, 31 (Suppl. 1), 88-91 (1999); and Lavanchy, J. Viral Hepatitis, 6, 35-47 (1999). HCV includes a nucleocapsid protein (C), envelope proteins (E1 and E2), and several non-structural proteins (NS2, NS3, NS4a, NS5a, and NS5b).

NS3 protein, which possesses serine protease activity, is considered essential for viral replication. This is evidenced by the observations that mutations in the yellow fever virus NS3 protease decreased viral infectivity and mutations at the active site of the HCV NS3 protease completely inhibited the HCV infection in a chimpanzee model. See, e.g., Chamber et al., Proc. Natl. Acad. Sci. USA 87, 8898-8902 (1990) and Rice et al., J. Virol. 74 (4) 2046-51 (2000). Further, the HCV NS3 serine protease was found to facilitate proteolysis at the NS3/NS4a, NS4a/NS4b, NS4b/NS5a, NS5a/NS5b junctions. It is therefore believed that the HCV NS3 serine protease is responsible for generating four viral proteins during viral replication. See, e.g., US 2003/0207861. Consequently, the HCV NS3 serine protease is an attractive target in treating HCV infection. Potential NS3 HCV protease inhibitors can be found in WO 02/18369, WO 00/09558, WO 00/09543, WO 99/64442, WO 99/07733, WO 99/07734, WO 99/50230, WO 98/46630, WO 98/17679, WO 97/43310, U.S. Pat. No. 5,990,276, Dunsdon et al., Biorg. Med. Chem. Lett. 10, 1571-1579 (2000); Llinas-Brunet et al., Biorg. Med. Chem. Lett. 10, 2267-2270 (2000); and S. LaPlante et al., Biorg. Med. Chem. Lett. 10, 2271-2274 (2000).

Currently, interferon-α, pegylated interferon-α, and a combination of interferon-α/ribavirin are the only anti-HCV therapeutic agents. However, sustained response rates for interferon-α or interferon-α/ribavirin have been found to be <50% and patients suffer greatly from side effects of these therapeutic agents. See, e.g., Walker, DDT, 4, 518-529 (1999); Weiland, FEMS Microbial. Rev., 14, 279-288 (1994); and WO 02/18369. Thus, there remains a need for developing more effective and receptive anti-HCV drugs.

### SUMMARY

This invention is based on the unexpected discovery that certain pyrrolidine compounds are effective in inhibiting an HCV protease.

In one aspect, this invention features pyrrolidine compounds of formula (I):

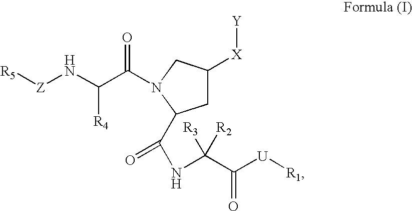

in which each of R₁, R₂, R₃ R4, and R₅, independently, is H, C₁₋₆ alkyl, C₁₋₆ alkoxyl, C₃₋₁₀ cycloalkyl, C₁₋₁₀ heterocycloalkyl, C₆₋₁₀ aryl, or C₃₋₁₀ heteroaryl; or R₂ and R₃, together with the carbon atom to which they are attached, form a C₃₋₁₀ cycloalkyl and C₁₋₁₀ heterocycloalkyl optionally having one or more substituents selected from a group consisting of halo, nitro, cyano, C₁₋₆ alkyl, C₁₋₆ alkoxyl, C₂₋₆ alkenyl, C₂₋₆ alkynyl, C₆₋₁₀ aryl, and C₃₋₁₀ heteroaryl; U is —O—, —NH—, —C(O)NH—, —NHSO—, or —NHSO₂—; X is —O—, —S—, —NH—, or —OCH₂—; Y is

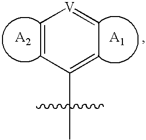

in which V is —CH—or —N—; and each of A₁ and A₂, independently, is selected from the group consisting of C₃₋₁₀ cycloalkyl, C₁₋₁₀ heterocycloalkyl, C₆₋₁₀ aryl, and C₃₋₁₀ heteroaryl, each of which is optionally substituted with halo, nitro, cyano, C₁₋₆ alkyl, C₁₋₆ alkoxyl, C₂₋₆ alkenyl, C₂₋₆ alkynyl, C₆₋₁₀ aryl, or C₃₋₁₀ heteroaryl, or optionally fused with another C₃₋₁₀ cycloalkyl, C₁₋₁₀ heterocycloalkyl, C₆₋₁₀ aryl, and C₃₋₁₀ heteroaryl, optionally substituted with halo, nitro, cyano, C₁₋₆ alkyl, C₁₋₆ alkoxyl, C₂₋₆ alkenyl, C₂₋₆ alkynyl, C₆₋₁₀ aryl, or C₃₋₁₀ heteroaryl; and Z is —C(O), —O—C(O)—, —NH—C(O)—, —O—C(S)—, —NH—C(S)—, —O—C(NH)—, or —NH—C(NH)—.

Referring to formula (I), the compounds described above may possess one or more of the following features: R₁ is cyclopropyl; R₂ and R₃, together with the carbon atom to which they are attached, form cyclopropyl (which may be substituted with vinyl); R4 is C₁₋₆ alkyl; R₅ is cyclopentyl; X is —O—; U is —NHSO₂—; Z is —OC(O)—; Ar₂ is phenyl; V is —N—; and Y is selected from

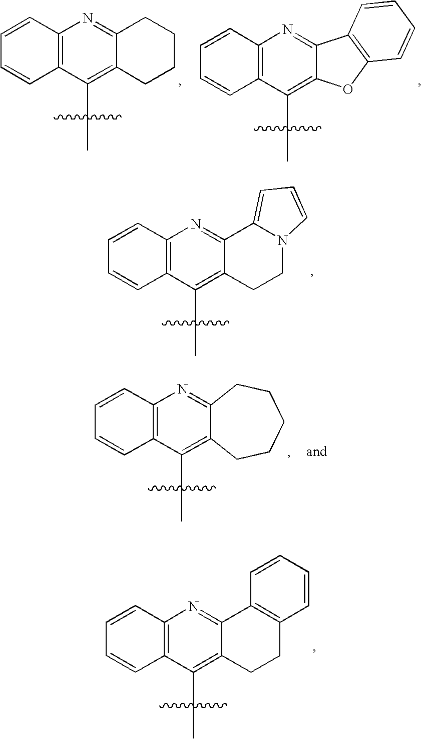

each of which is optionally substituted with halo, C₁₋₆ alkyl, or C₁₋₆ alkoxyl.

Further, the compounds described above may have the stereochemistry as shown below:

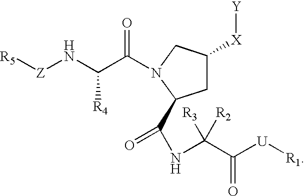

The term “alkyl” refers to a straight or branched hydrocarbon, containing 1-10 carbon atoms. Examples of alkyl include, but are not limited to, methyl, ethyl, n-propyl, i-propyl, n-butyl, i-butyl, and t-butyl. The term “alkenyl” refers to a straight or branched hydrocarbon containing 2-10 carbon atoms and one or more double bonds. Examples of alkenyl, but are not limited to, include vinyl, propenyl, allyl, and 1,4-butadienyl. The term “alkynyl” refers to a straight or branched hydrocarbon containing 2-10 carbon atoms and one or more triple bonds. Examples of alkynyl include, but are not limited to, ethynyl, 1-propynyl, 1- and 2-butynyl, and 1-methyl-2-butynyl. The term “alkoxy” refers to an —O-alkyl radical. Examples of alkoxy include, but are not limited to, methoxy, ethoxy, n-propoxy, isopropoxy, n-butoxy, iso-butoxy, sec-butoxy, and tert-butoxy.

The term “cycloalkyl” refers to a saturated, cyclic hydrocarbon moiety, such as cyclohexyl. The term “cycloalkenyl” refers to a non-aromatic, cyclic hydrocarbon moiety that contains at least one double bond, such as cyclohexenyl. The term “heterocycloalkyl” refers to a saturated, cyclic moiety having at least one ring heteroatom (e.g., N, O, or S), such as 4-tetrahydropyranyl. The term “heterocycloalkenyl” refers to a non-aromatic, cyclic moiety having at least one ring heteroatom (e.g., N, O, or S) and at least one ring double bond, such as pyranyl. The term “aryl” refers to a hydrocarbon moiety having one or more aromatic rings. Examples of aryl moieties include phenyl, phenylene, naphthyl, naphthylene, pyrenyl, anthryl, and phenanthryl. The term “heteroaryl” refers to a moiety having one or more aromatic rings that contain at least one heteroatom (e.g., N, O, or S). Examples of heteroaryl moieties include furyl, furylene, fluorenyl, pyrrolyl, thienyl, oxazolyl, imidazolyl, thiazolyl, pyridyl, pyrimidinyl, quinazolinyl, quinolyl, isoquinolyl, and indolyl.

Alkyl, alkenyl, alkynyl, cycloalkyl, cycloalkenyl, heterocycloalkyl, heterocycloalkenyl, aryl, and heteroaryl mentioned herein include both substituted and unsubstituted moieties, unless specified otherwise. Possible substituents on cycloalkyl, cycloalkenyl, heterocycloalkyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, C₁-C10 alkylsulfonamino, arylsulfonamino, C₁-C₁₀ alkylamino, arylamino, C₁-C₁₀ alkylsulfonamino, arylsulfonamino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, nitroso, azido, acyl, thioacyl, acyloxy, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

The compounds of formula (I) described above include the compounds themselves, as well as their salts, prodrugs, and solvates, if applicable. A salt, for example, can be formed between an anion and a positively charged group (e.g., amino) on a compound of formula (I). Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a compound of formula (I). Suitable cations include sodium ion, potassium ion, magnesium ion, calcium ion, and an ammonium cation such as tetramethylammonium ion. The compounds of formula (I) also include those salts containing quaternary nitrogen atoms. Examples of prodrugs include esters and other pharmaceutically acceptable derivatives, which, upon administration to a subject, are capable of providing active compounds of formula (I). A solvate refers to a complex formed between an active compound of formula (I) and a pharmaceutically acceptable solvent. Examples of pharmaceutically acceptable solvents include water, ethanol, isopropanol, ethyl acetate, acetic acid, and ethanolamine.

The compounds may also contain a non-aromatic double bond and one or more asymmetric centers. Thus, they can occur as racemates and racemic mixtures, single enantiomers, individual diastereomers, diastereomeric mixtures, tautomers, and cis- or trans- isomeric forms. All such isomeric forms are contemplated.

Shown below are 38 exemplary compounds of this invention:

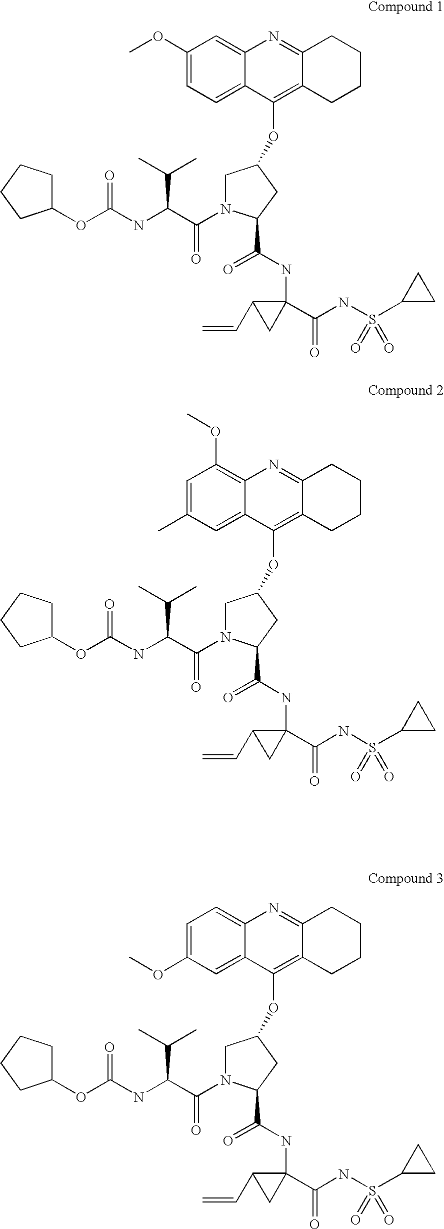

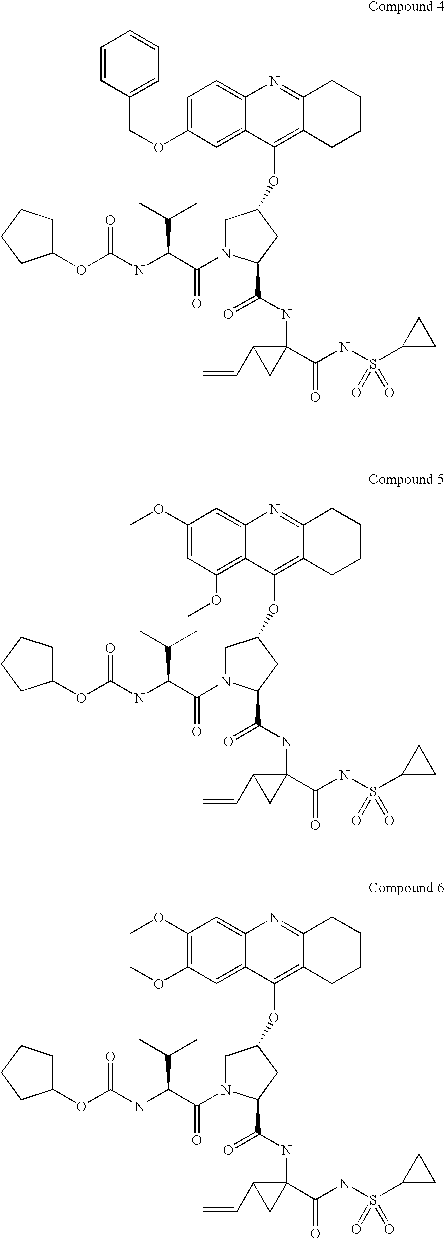

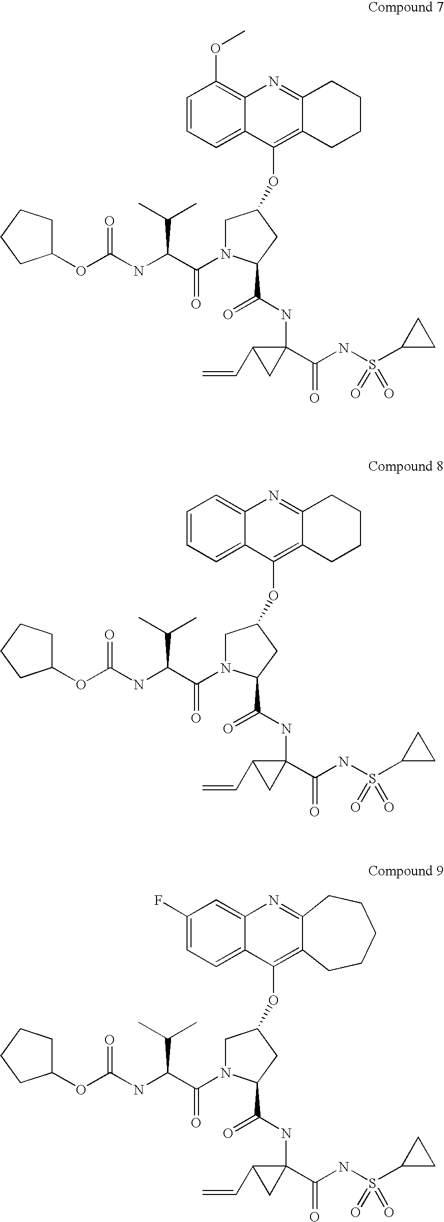

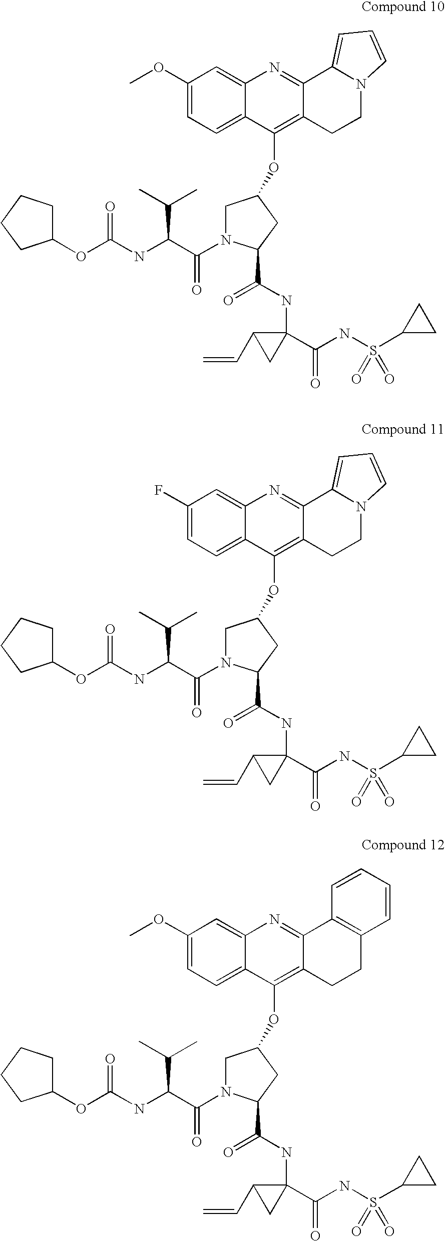

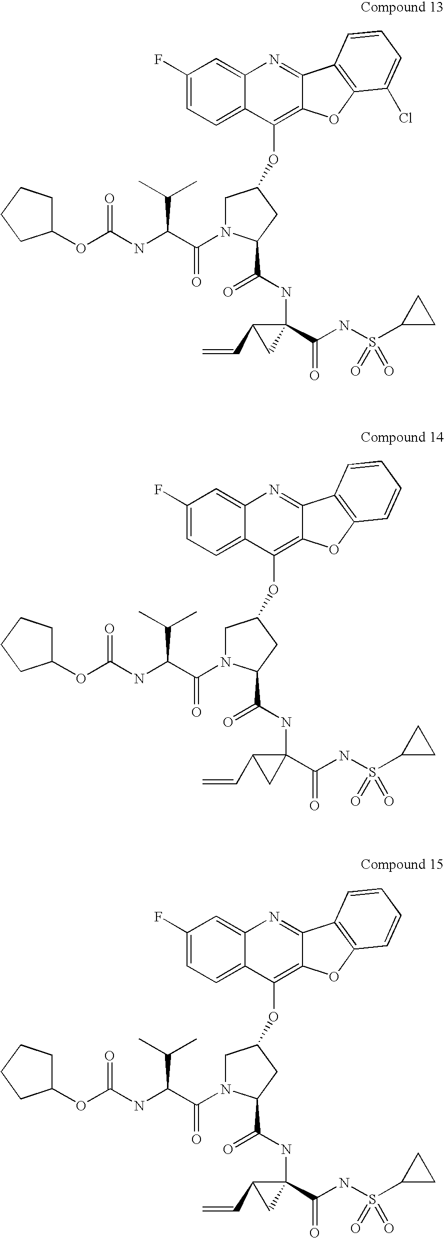

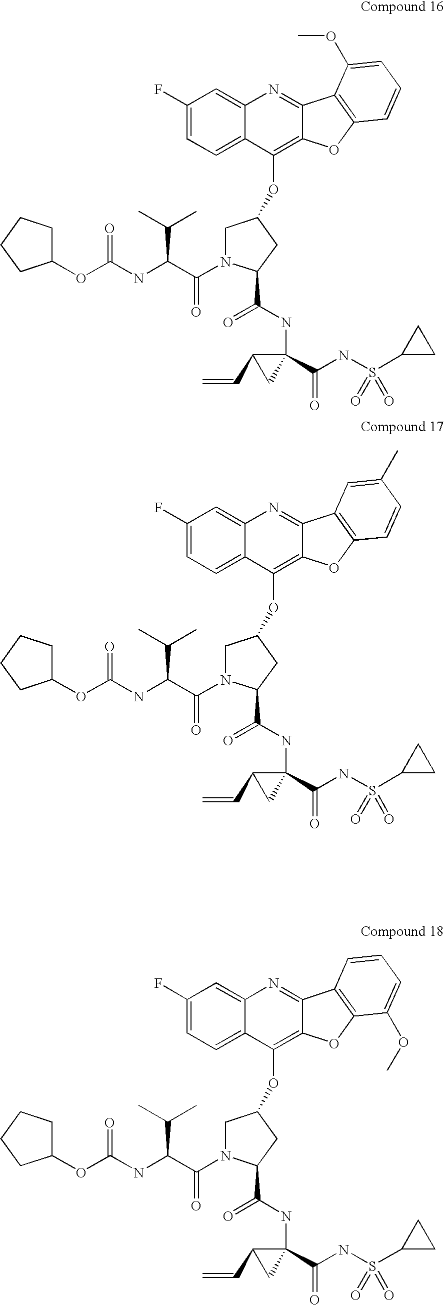

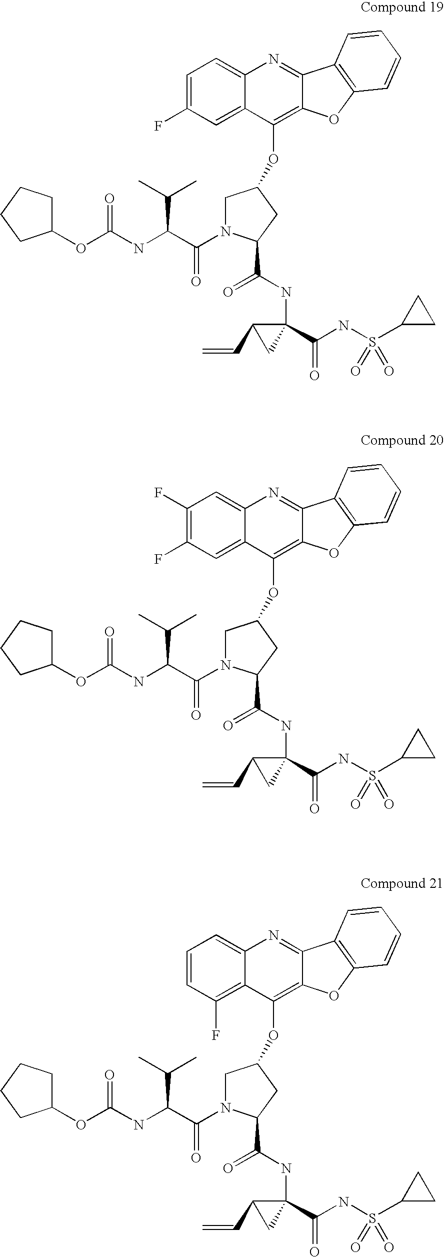

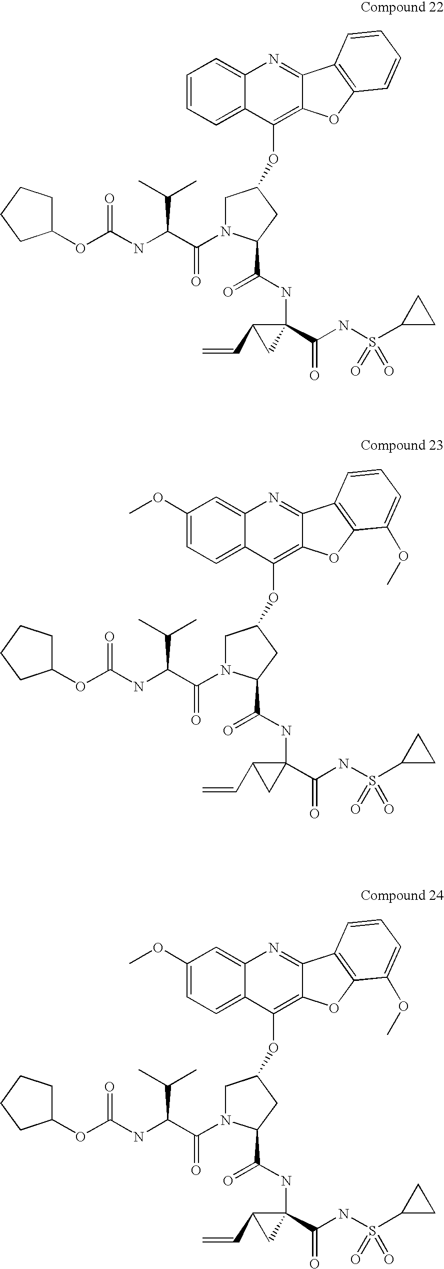

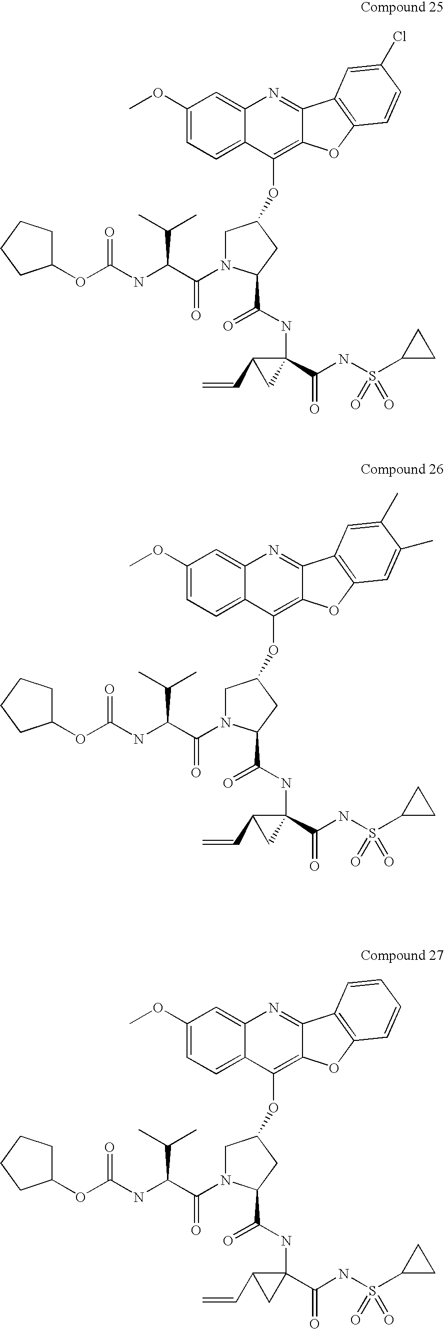

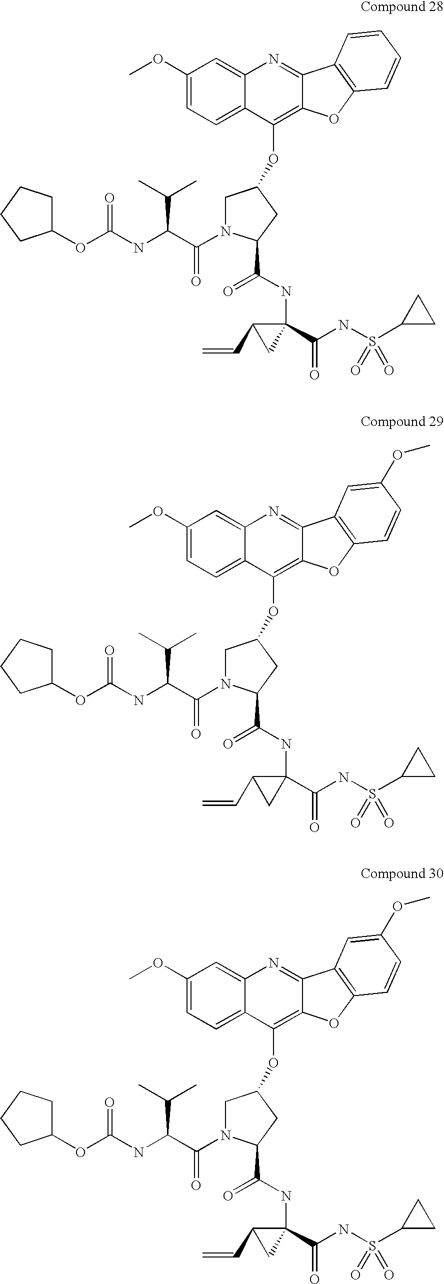

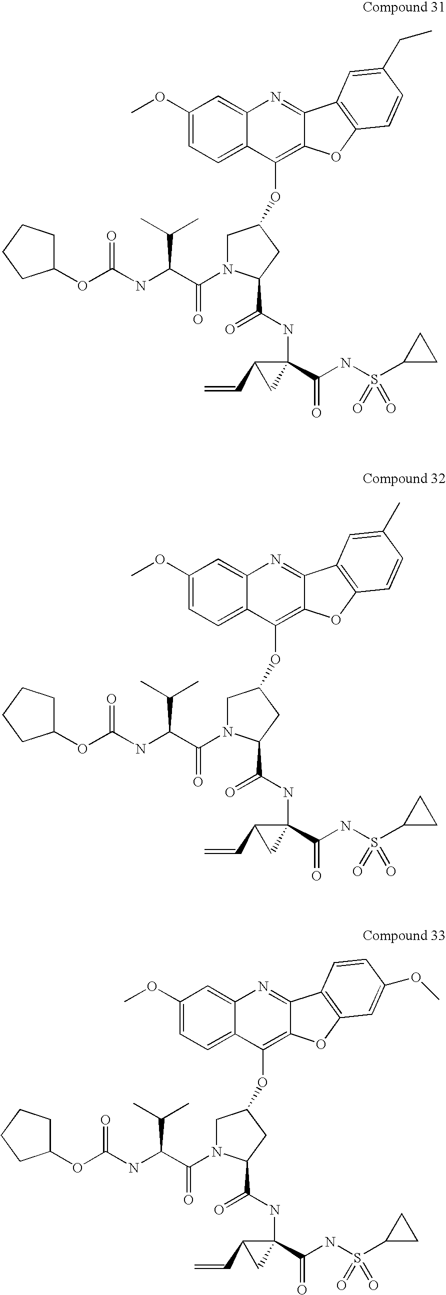

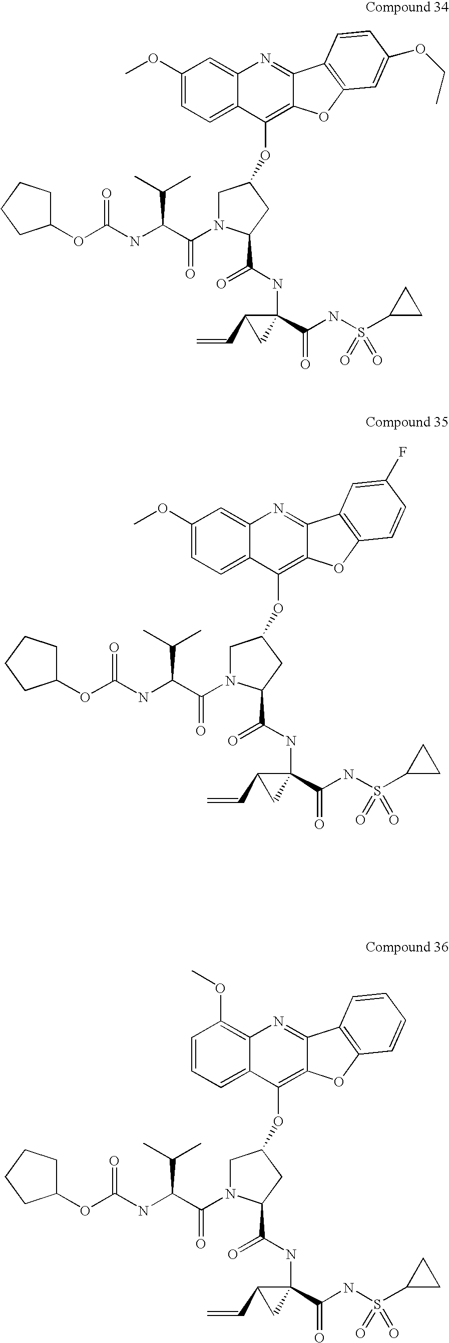

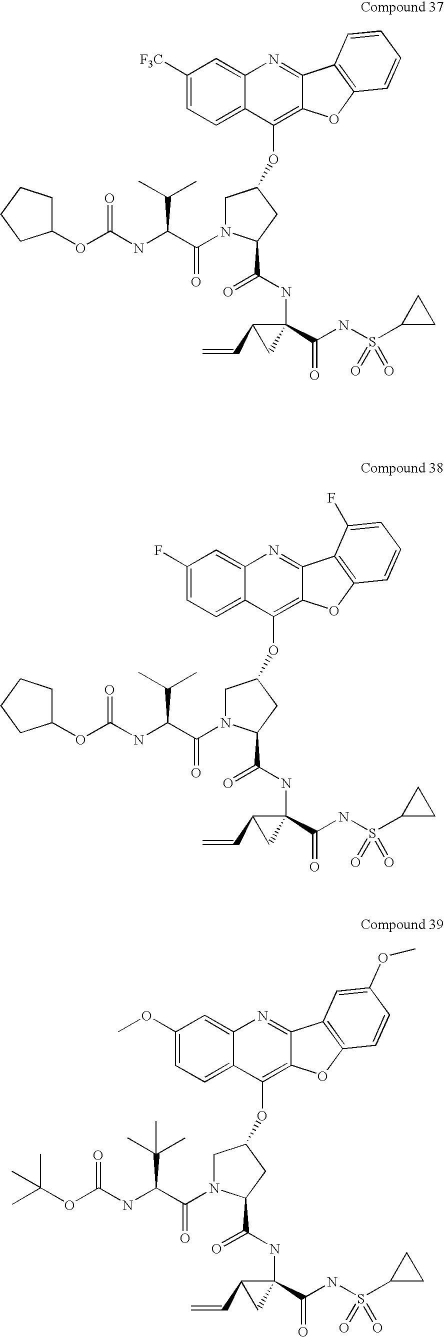

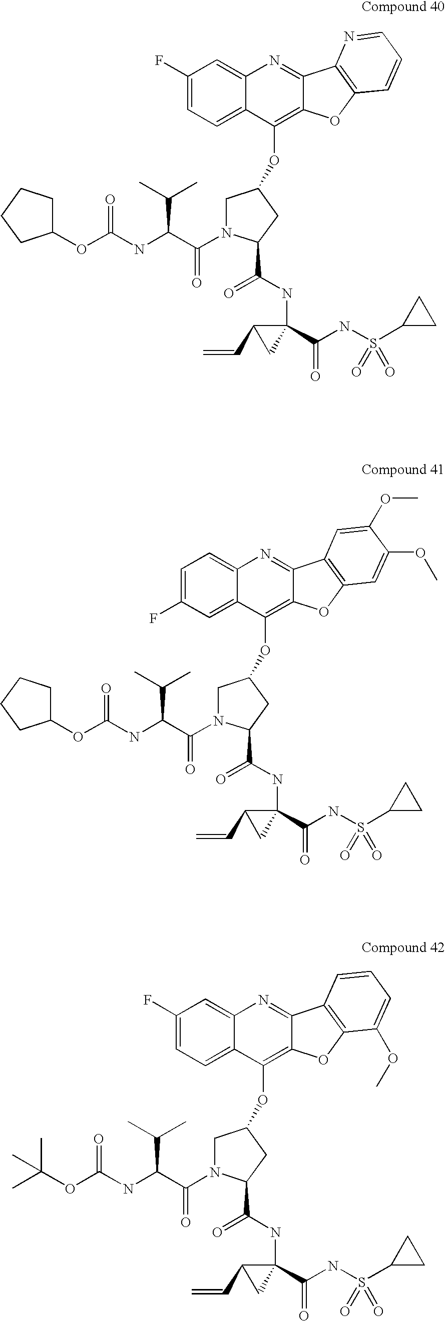

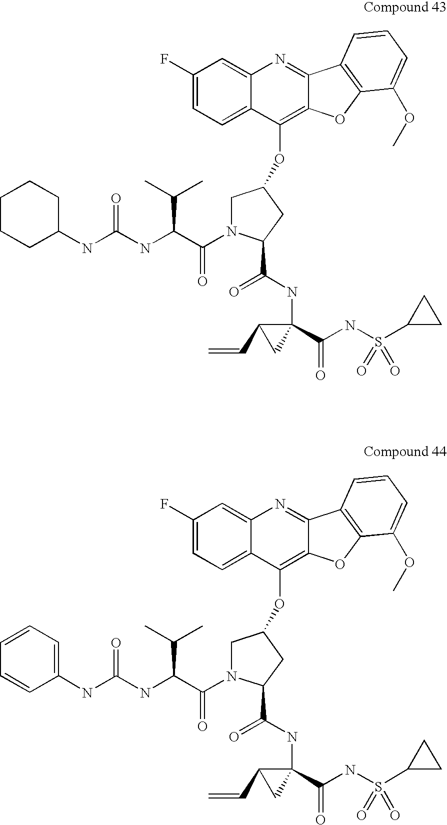

In another aspect, this invention features a method for treating HCV infection by administering an effective amount of one or more of the pyrrolidine compounds of formula (I) to a patient infected with HCV.

Also within the scope of this invention is a pharmaceutical composition containing one or more of the pyrrolidine compounds of formula (I) for use in treating HCV infection, as well as this use and use of one or more of the pyrrolidine compounds for the manufacture of a medicament for the just-mentioned treatment.

The details of one or more embodiments of the invention are set forth in the description below. Other features, objects, and advantages of the invention will be apparent from the description and from the claims.

### DETAILED DESCRIPTION

The pyrrolidine compounds of the present invention can be prepared by methods well known in the art. Scheme 1 below illustrates a typical route for synthesizing certain pyrrolidine compounds of this invention:

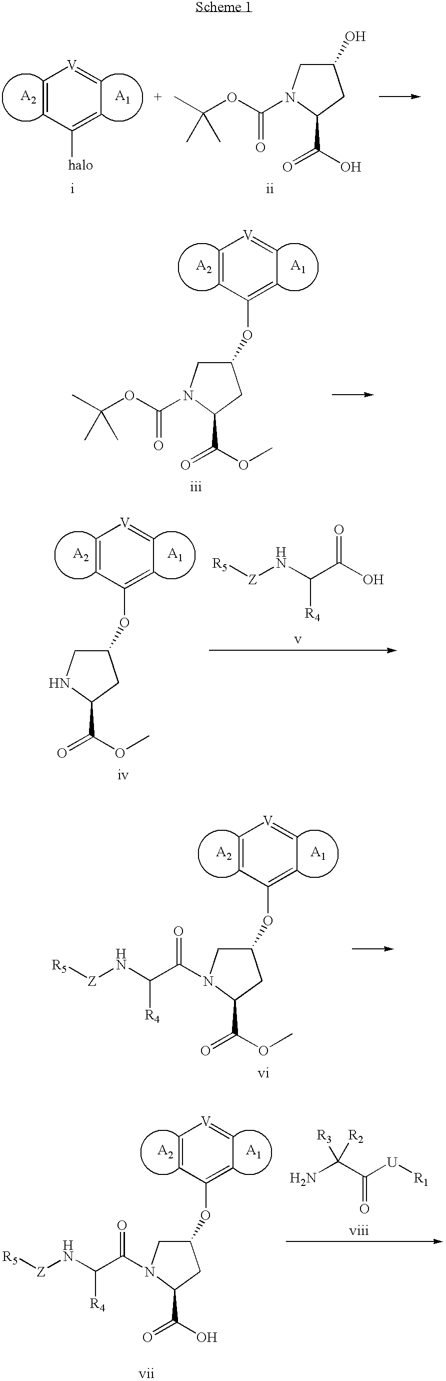

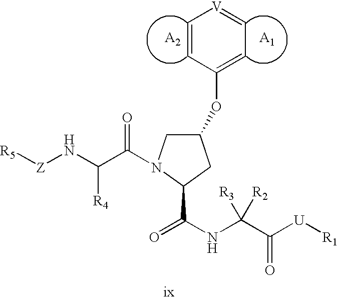

A brief description of the reactions shown in scheme 1 follows:

Multicyclic compound (i) is coupled with N-(t-butoxycarbonyl)-L-proline (ii), followed by methylation, to form intermediate (iii). Intermediate (iii) is deprotected by removing the N-butoxycarbonyl group to produce N-free compound (iv), which is coupled with carboxylic acid (v) to afford intermediate (vi). Intermediate (vi) is deprotected by hydrolyzing the methyl carboxylate group to give acid (vii), which is coupled with amine compound (viii) to provide desired pyrrolidine compound (ix).

The starting materials used in the above synthetic route are either commercially available or can be readily made according to methods already reported. Synthetic chemical transformations and protecting group methodologies (protection and deprotection) required in the above synthetic route are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

A compound synthesized above can be purified by a suitable method such as column chromatography, high-pressure liquid chromatography, or recrystallization.

Examples 1-38 below provide detailed descriptions of how compounds 1-38 were actually prepared.

Also within the scope of this invention is a pharmaceutical composition containing an effective amount of at least one pyrrolidine compound of formula (I) and a pharmaceutical acceptable carrier. Further, this invention covers a method of treating HCV infection by administering an effective amount of one or more of the compounds of formula (I) to a patient infected with HCV. The term “treating” or “treatment” refers to administering one or more compounds of formula (I) to a subject, who has HCV infection, a symptom of it, or a predisposition toward it, with the purpose to confer a therapeutic effect, e.g., to cure, relieve, alter, affect, ameliorate, or prevent the HCV infection, the symptom of it, or the predisposition toward it. The term “an effective amount” refers to the amount of an active compound of formula (I) that is required to confer a therapeutic effect on the treated subject. Effective doses will vary, as recognized by those skilled in the art, depending on the types of diseases treated, route of administration, excipient usage, and the possibility of co-usage with other therapeutic treatment.

To practice the method of the present invention, a pharmaceutical composition containing at least one pyrrolidine compound of formula (I) and a pharmaceutical acceptable carrier can be administered parenterally, orally, nasally, rectally, topically, or buccally. The term “parenteral” as used herein refers to subcutaneous, intracutaneous, intravenous, intrmuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more pyrrolidine compounds of formula (I) can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound of formula (I). Examples of other carriers include colloidal silicon oxide, magnesium stearate, cellulose, sodium lauryl sulfate, and D&C Yellow #10.

The compounds of this invention can be used together with one or more other active agents to treat HCV infection. Thus, this invention also relates to a method of treating HCV infection by administering to a subject in need of the treatment an effective amount of a compound of this invention and effective amounts of one or more other active agents. Active agents include, but are not limited to, immunomodulatory agents, such as interferons α, β, and γ; antiviral agents such as ribavirin and amantadine; other inhibitors of HCV NS3 protease; inhibitors of other targets in the HCV life cycle such as the helicase, polymerase, metalloprotease, or internal ribosome entry site. Such an active agent and a compound of this invention may be applied to a subject at two separate times or simultaneously but in two dosage forms. Alternatively, they can be combined in a composition as described above for use as a single dosage form.

The compounds of this invention described above can be preliminarily screened for their efficacy in inhibiting HCV protease by an in vitro assay (Example 39 below). The compounds can further be examined to verify their efficacy in treating HCV infection. For example, a compound can be administered to an animal (e.g., a mouse model) infected with HCV and its therapeutic effects are then assessed. Based on the results, an appropriate dosage range and administration route can also be determined.

Without further elaboration, it is believed that the above description has adequately enabled the present invention. The following examples are, therefore, to be construed as merely illustrative, and not limitative of the remainder of the disclosure in any way whatsoever. All of the publications cited herein are hereby incorporated by reference in their entirety.

### EXAMPLE 1

### Synthesis of cyclopentyl (2S)-1-((2S,4R)-2-(1-(cyclopropylsulfonylcarbamoyl)-2-vinylcyclopropylcarbamoyl)-4-(6-methoxy-1,2,3,4-tetrahydroacridin-9-yloxy)pyrrolidin-1-yl)-3-methyl-1-oxobutan-2-ylcarbamate

### (Compound 1)

Compound I-3 useful for synthesizing compound 1 was prepared from commercially available 2-amino-3-methyl-butyric acid methyl ester via the route shown below.

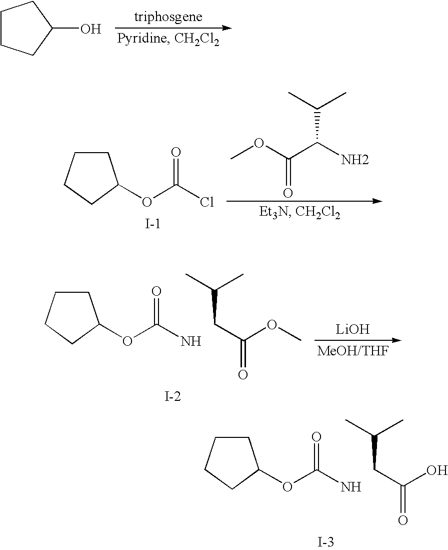

To a solution of 1-cyclopantanol (1.72 g, 20.0 mmol) and pyridine (2.37 g, 30.0 mmol) in CH₂Cl₂ (80 mL) was added triphosgene (2.18 g, 22.0 mmol) at 0° C. The reaction mixture was warmed to room temperature and stirred for additional 3 hours. It was then quenched with 10% HCl and subjected to extraction with dichloromethane (60 mL×2). The organic layer was collected, dried over MgSO₄, and concentrated under vacuum to obtain crude compound I-1.

To Et₃N (3.03 g, 30.0 mmol) was added dropwise a solution of crude compound I-1 (1.63 g, 11.0 mmol) and 2-amino-3-methyl-butyric acid methyl ester (2.43 g, 10.0 mmol) in CH₂Cl₂ (50 mL) at 0° C. After stirred for 3 hours, the reaction mixture was acidified by 10% HCl to pH <7 and concentrated under vacuum. The residue was filtered and washed with water to give compound I-2 (2.26 g, 93%). MS m/z 244.1 (M⁺+1); ¹H NMR (CDCl₃) δ 5.13-5.02 (m, 2H), 4.25 (dd, J=9.2 Hz, J=4.5 Hz, 1H), 3.50 (s, 3H), 2.17-2.05 (m, 1H), 1.88-1.49 (m, 8H), 0.93 (d, J=6.6 Hz, 3H) 0.86 (d, J=6.9 Hz, 3H).

To a solution of compound I-2 (2.43 g, 10.0 mmol) in THF (50 mL) was added 0.5 M LiOH (60 mL, 30.3 mmol) at room temperature. After stirred overnight, the reaction mixture was acidified by 10% HCl to pH <7 and concentrated under vacuum. The residue was filtered and washed with water to give compound I-3 (2.13 g, 93%). MS: m/z 230.1 (M⁺+1); ¹H NMR (CDCl₃) δ: 5.12-5.04 (m, 2H), 4.28 (dd, J=9.3 Hz, J=4.6 Hz, 1H), 2.15-2.10 (m, 1H), 1.86-1.45 (m, 8H), 0.95 (d, J=6.6 Hz, 3H), 0.87 (d, J=6.9 Hz, 3H).

Compound I-6 also useful for synthesizing Compound 1 was prepared from commercially available 1-tert-butoxycarbonylamino-2-vinyl-cyclopropanecarboxylic acid ethyl ester via the route shown below:

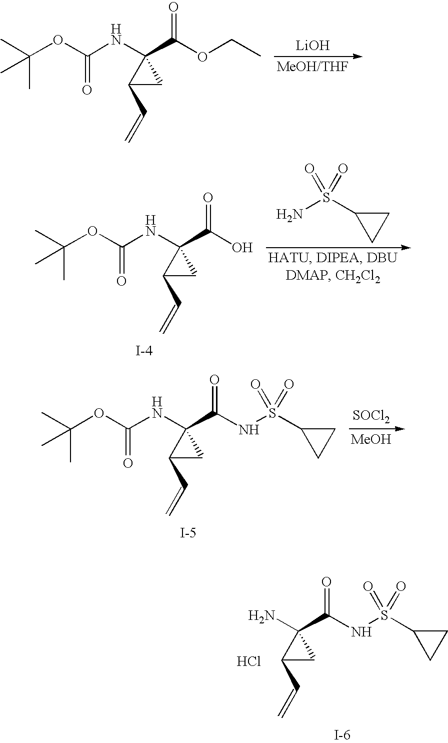

To a solution of 1-tert-butoxycarbonylamino-2-vinyl-cyclopropanecarboxylic acid ethyl ester (0.34 g, 1.3 mmol) in THF (5 mL) and methanol (5 mL) was added a suspension of LiOH (0.13 g, 5.3 mmol) in water (1.4 mL). After stirred overnight at room temperature, the reaction was quenched with 10% HCl (2 mL) and the solvent was removed under vacuum. The resultant solid powder was washed with water (10 mL) to give compound I-4 (0.27 g, 90%). MS m/z 249.9 (M⁺+23); ¹H NMR (CDCl₃) δ 10.35 (brs, 1H), 5.84-5.71 (m, 1H), 5.29 (d, J=17.4 Hz, 1H), 5.12 (d, J=10.2 Hz, 1H), 2.23-2.14 (m, 1H), 1.87-1.65 (m, 1H), 1.58-1.41 (m, 1H), 1.43 (s, 9H).

A solution of compound I-4 (0.52 g, 2.3 mmol), 2-(1H-7-azabenzotriazol-1-yl)-1,1,3,3-tetramethyl uronium hexafluoro-phosphate methanaminium (HATU, 1.74 g, 4.6 mmol), and 4-dimethylaminopyridine (1.39 g, 11.6 mmol) in CH₂Cl₂ (40 mL) was stirred at room temperature for 1 hour, followed by slow addition of cyclopropanesulfonamide (0.57 g, 4.7 mmol), diisopropylethylamine (1.81 mL, 14.0 mmol) and 1,8-diazabicyclo[5,4,0]undec-7-ene (1.80 g, 11.7 mmol) over 15 minutes. After the reaction mixture was stirred at room temperature overnight, the solvent was removed under vacuum. The residue was purified by silica gel column chromatography to give compound I-5 (0.51 g, 66%). MS m/z 353.1 (M⁺+23); ¹H NMR (CDCl₃) δ 9.75 (brs, 1H), 5.64-5.51 (m, 1H), 5.30 (d, J=17.4 H), 5.16 (d, J=10.2 Hz, 1H), 2.95-2.89 (m, 1H), 2.19-2.10 (m, 1H), 1.93-1.88 (m, 1H), 1.47 (s, 9H), 1.46-1.38 (m, 1H), 1.32-1.23 (m, 2H), 1.15-1.00 (m, 2H).

To a solution of compound I-5 (0.50 g, 1.5 mmol) in MeOH (8 mL) was added SOCl₂ (0.26 g, 2.2 mmol) at room temperature. After the reaction mixture was refluxed for 1 hour, MeOH and SOCl₂ was removed under vacuum. The residue was triturated from pentane and filtered to give intermediate I-6 as an off-white solid (0.32 g, 91%). MS m/z (M⁺+1); ¹H NMR (CD₃COD) δ 5.77-5.65 (m, 1H), 5.43 (d, J=17.4 Hz, 1H), 5.32 (d, J=10.2 Hz, 1H), 3.06-2.97 (m, 1H), 2.45 (dd, J=17.4 Hz, J=7.8, 1H, 2.16 (dd, J=8.0 Hz, J=7.8 Hz, 1H), 1.75 (dd, J=10.1 Hz, J=7.8 Hz, 1H), 1.32-0.86 (m, 4H).

Compound 1 was prepared via the route shown in the scheme below:

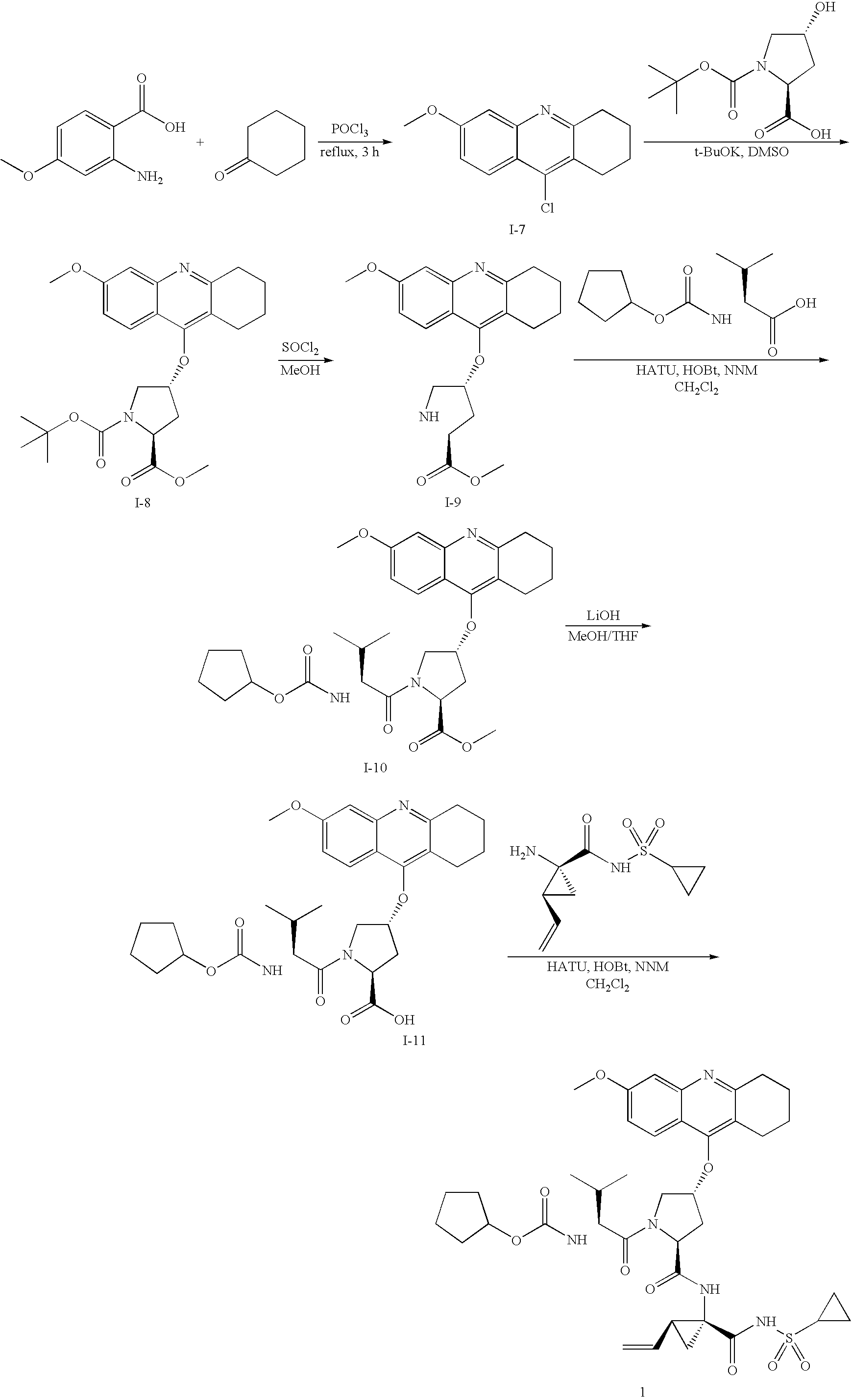

A solution of 2-amino-4-methoxy-benzoic acid (1.67 g, 10.0 mmol) and 1-hexanone (118 g, 12.0 mmol) in excess phosphorus oxychloride (POCl₃) was refluxed for 3 hours. After cooled and thoroughly concentrated, the residue was subjected to extraction with methylene chloride and 10% sodium hydroxide. The organic layer was dried over MgSO₄, concentrated, and purified by silica gel column chromatography to give compound I-7 (1.69 g, 71%). MS m/z 248.1 (M⁺+1); ¹H NMR (CDCl₃) δ 7.56 (d, J=9.0 Hz, 1H), 7.14 (d, J=2.7 Hz, 1H), 6.90 (dd, J=9.0, J=2.7 Hz, 1H), 3.81 (s, 3H), 2.86 (brs, 2H), 2.67 (brs, 2H), 1.82-1.71 (m, 4H).

To a suspension of boc-trans-4-hydroxy-L-proline (1.90 g, 8.2 mmol) in DMSO (25 mL) was added t-BuOK (1.84 g, 16.4 mmol) at 0° C. After the reaction mixture was warmed to room temperature and stirred for 1 hour, compound I-7 (2.05 g, 8.3 mmol) was added in one portion. Stirring was continued overnight and iodomethane (1.40 g, 9.84 mmol) was added. After stirred at room temperature for additional 30 minutes, the mixture was acidified to pH 6˜7 by using 10% HCl aqueous solution and subjected to extraction with methylene chloride. The organic layer was dried over MgSO₄, evaporated under vacuum, and purified by silica gel column chromatography to give compound I-8 (2.73 g, 73%). MS m/z 457.1 (M⁺+1).

To a solution of compound I-8 (0.46 g, 1.1 mmol) in MeOH (8 mL) was added SOCl₂ (0.39 g, 3.3 mmol) at room temperature. The reaction mixture was refluxed for 1 hour, and then MeOH and SOCl₂ were removed under vaccum. The residue was triturated in pentane and filtered to give compound I-9 as an off-white solid (0.35 g, 90%). MS m/z 357.2 (M⁺+1).

N-Methylmorpholine (NMM, 2.64 g, 26.1 mmol) was added to a solution of compound I-9 (2.16 g, 5.2 mmol), HATU (2.97 g, 7.8 mmol), N-Hydroxybenzotriazole (HOBT, 1.08 g, 7.8 mmol), and compound I-3 (1.19 g, 5.2 mmol) in CH₂Cl₂ (40 mL) at room temperature. After stirred overnight, the mixture was concentrated under vacuum. The residue was purified by silica gel column chromatography to give compound I-10 (2.70 g, 83%). MS m/z 568.3 (M⁺+1).

To a solution of compound I-10 (1.13 g, 2.0 mmol) in THF (50 mL) was added 0.5 M LiOH (20 mL, 10.1 mmol) at room temperature. After stirred overnight, the reaction mixture was acidified by 10% HCl to pH <7 and concentrated under vacuum to give a solid, which was filtered and washed with water to give compound I-11 (1.03 g, 93%). MS: m/z 554.0 (M⁺+1); ¹H NMR (CDCl₃) δ: 7.71 (d, J=9.3 Hz, 1H), 7.18 (s, 1H), 7.03 (dd, J=9.3, 2.7 Hz, 1H), 5.93 (brs, 1H), 5.34-5.28 (m, 1H), 4.96 (brs, 1H), 4.83 (dd, J=8.1, J=7.8 Hz, 1H), 4.33-4.02 (m, 3H), 3.93 (s, 3H), 3.01-2.97 (m, 2H), 2.64-2.59 (m, 4H), 2.09-2.03 (m, 1H), 1.90-1.51 (m, 13H), 1.02 (d, J=6.6 Hz, 3H), 0.95 (d, J=6.9 Hz, 3H).

A solution of compound I-11 (0.23 g, 0.41 mmol), HATU (0.31 g, 0.81 mmol), HOBT (0.08 g, 0.61 mmol) and compound I-7 (0.09 g, 0.41 mmol) in CH₂Cl₂ (10 mL) was added NMM (0.12 g, 1.21 mmol) at room temperature. After stirred overnight, the reaction mixture was concentrated under vacuum and purified by silica gel column chromatography to give compound 1 (0.22 g, 65%). MS m/z 766.4 (M⁺+1); ¹H NMR (CDCl₃) δ 7.69 (d, J=9.0 Hz, 1H), 7.14 (s, 1H), 7.16 (d, J=9.0 Hz, 1H), 5.94-5.78 (m, 3H), 5.37-4.93 (m, 3H), 4.52-4.42 (m, 1H), 4.29-4.01 (m, 3H), 3.92 (s, 3H), 2.98-2.78 (m, 3H), 2.63-2.32 (m, 4H), 2.22-0.82 (m, 28H).

### EXAMPLES 2-12

### Syntheses of Compounds 2-12

Compounds 2-12 were synthesized in manners similar to that described in Example 1.

Compound 2: MS m/z 780.1 (M⁺+1).

Compound 3: MS m/z 766.3 (M⁺+1).

Compound 4: MS m/z 842.4 (M⁺+1).

Compound 5: MS m/z 795.8 (M⁺+1).

Compound 6: MS m/z 796.1 (M⁺+1); ¹H NMR (CDCl₃) δ 7.29 (s, 1H), 7.02 (s, 1H), 5.92-5.53 (m, 1H), 5.38-4.91 (m, 4H), 4.51 (brs, 1H), 4.37-4.21 (m, 2H), 3.97 (s, 3H), 3.91 (s, 3H), 3.05-2.63 (m, 5H), 2.45-0.81 (m, 31H).

Compound 7: MS m/z 766.3 (M⁺+1).

Compound 8: MS m/z 736.2 (M⁺+1).

Compound 9: MS m/z 768.3 (M⁺+1).

Compound 10: MS m/z 766.4 (M⁺+1); ¹H NMR (CDCl₃) δ 7.68 (d, J=8.7 Hz, 1H), 7.38 (s, 1H), 7.18-7.07 (m, 2H), 6.81 (s, 1H), 6.32 (s, 1H), 5.92-5.76 (m, 1H), 571-5.57 (m, 1H), 5.36-5.05 (m, 4H), 4.61-4.45 (m, 1H), 4.37-4.08 (m, 4H), 3.95 (s, 3H), 3.90-3.81 (m, 1H), 3.22 (brs, 2H), 3.01-2.84 (m, 2H), 2.53-1.98 (m, 4H), 1.97-0.84 (m, 20H).

Compound 11: MS: m/z 803.3 (M⁺+1).

Compound 12: MS: m/z 791.0 (M⁺+1).

### EXAMPLE 13

### Synthesis of cyclopentyl (S)-1-((2S,4R)-4-(9-chloro-3-fluorobenzofuro[3,2-b]quinolin-11-yloxy)-2-((1R,2S)-1-(cyclopropylsulfonylcarbamoyl)-2-vinylcyclopropylcarbamoyl)pyrrolidin-1-yl)-3-methyl-1-oxobutan-2-ylcarbamate

### (Compound 13)

Compound 13 was prepared via the route shown below:

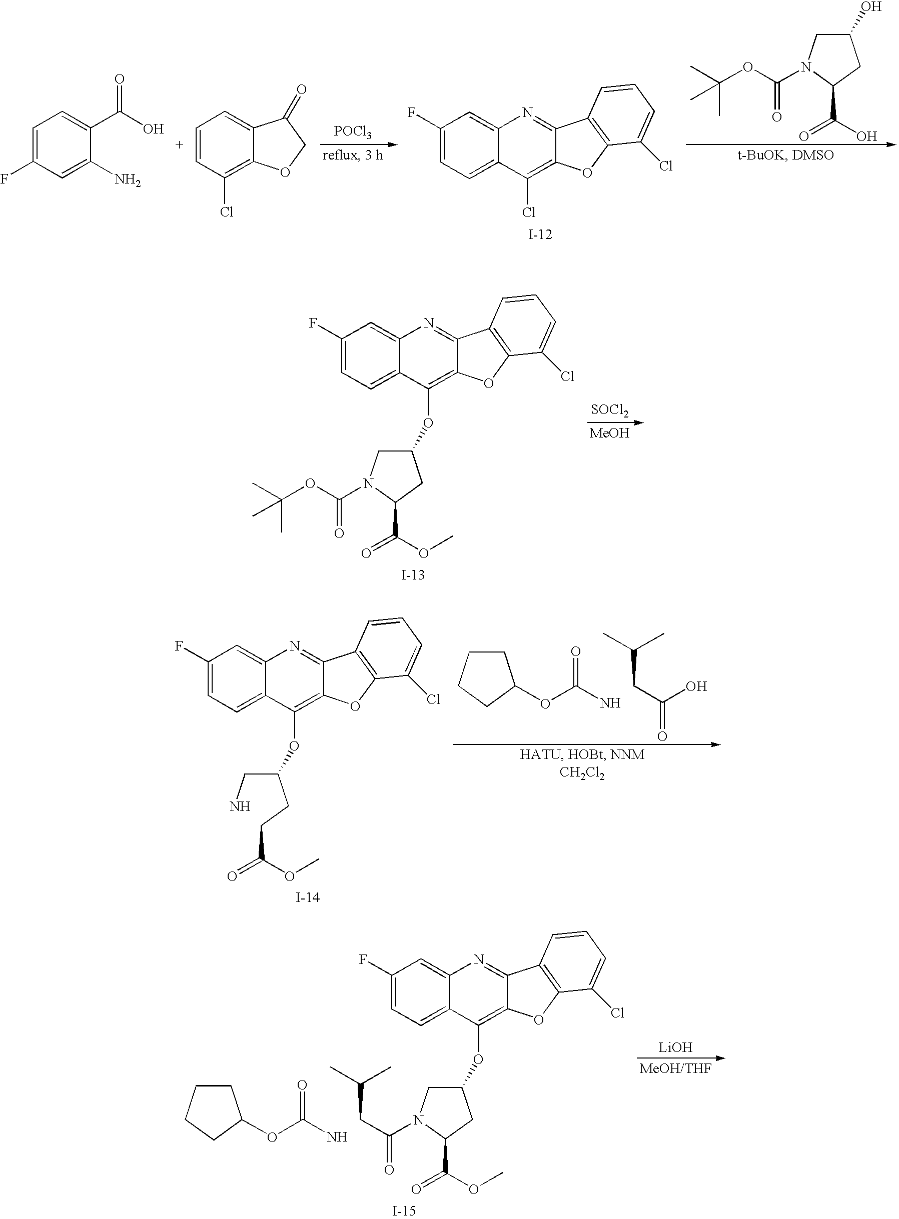

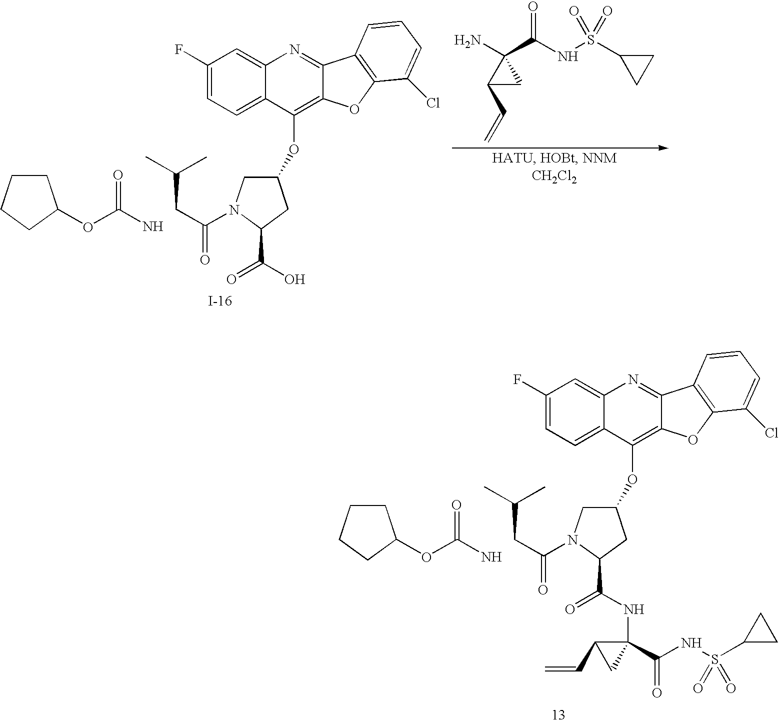

A solution of 2-amino-4-fluoro-benzoic acid (5.00 g, 32.2 mmol) and 7-chloro-benzofuran-3-one (4.41 g, 33.0 mmol) in excess phosphorus oxychloride (POCl₃) was refluxed for 3 hours. After cooled and thoroughly concentrated, the mixture was subjected to extraction with methylene chloride and 10% sodium hydroxide. The organic layer was dried over MgSO₄, concentrated, and purified by silica gel column chromatography to give compound I-12 (1.60 g, 18.3%). MS m/z 306.0, 308.0 (M⁺+1); ¹H NMR (CDCl₃) δ 8.36-8.31 (m, 2H), 7.93 (dd, J=10.1 Hz, J=2.7 Hz, 1H), 7.72-7.66 (m, 2H), 7.53-7.45 (m, 2H).

To a suspension of boc-trans-4-hydroxy-L-proline (1.91 g, 8.2 mmol) in DMSO (25 mL) was added t-BuOK (1.8 g, 16.4 mmol) at 0° C. After the mixture was allowed to warm to room temperature and stirred for hour, compound I-12 (2.51 g, 8.2 mmol) was added in one portion. Stirring was continued overnight. Iodomethane (1.40 g, 9.84 mmol) was added and the reaction mixture was stirred at room temperature for additional 30 minutes. The reaction mixture was acidified to pH 6˜7 by using 10% HCl aqueous solution and subjected to extraction with methylene chloride. The organic layer was dried over MgSO₄, evaporated under vacuum, and purified by silica gel column chromatography to give compound I-13 (3.08 g, 73%). MS m/z 515.0, 517.0 (M⁺+1).

To a solution of compound I-13 (1.02 g, 2.0 mmol) in MeOH (8 mL) was added SOCl₂ (0.71 g, 6.1 mmol) at room temperature. The reaction mixture was refluxed for 1 hour, and MeOH and SOCl₂ were removed. The residue was triturated in pentane and filtered to give compound I-14 as an off-white solid (0.76 g, 92%). MS m/z 415.1, 417.1 (M⁺+1).

NMM (2.64 g, 26.1 mmol) was added to a solution of compound I-14 (2.16 g, 5.2 mmol), HATU (2.97 g, 7.8 mmol), HOBT (1.08 g, 7.8 mmol) and I-3 (1.19 g, 5.2 mmol) in CH₂Cl₂ (40 mL) at room temperature. After stirred overnight, the mixture was concentrated under vacuum. The residue was purified by silica gel column chromatography to give compound I-15 (2.70 g, 83%). MS m/z 626.2, 628.2 (M⁺+1).

To a solution of compound I-15 (1.25 g, 2.0 mmol) in THF (50 mL) was added 0.5 M LiOH (20 mL, 10.1 mmol) at room temperature. After stirred overnight, the reaction mixture was acidified by 10% HCl to pH <7 and concentrated under vacuum. The resultant solid was filtered and washed by water to give compound I-16 (1.11 g, 91%). MS: m/z 612.0, 614.0 (M⁺+1).

NMM (0.12 g, 1.2 mmol) was added to a solution of compound I-16 (0.25 g, 0.4 mmol), HATU(0.31 g, 0.8 mmol), HOBT (0.08 g, 0.6 mmol) and compound I-6 (0.09 g, 0.4 mmol) in CH₂Cl₂ (10 mL) at room temperature. After stirred overnight, the reaction mixture was concentrated under vacuum. The residue was purified by silica gel column chromatography to give compound 13 (0.22 g, 65%). MS m/z 824.3 (M⁺+1); ¹H NMR (CDCl₃) δ 8.31-8.22 (m, 2H), 7.73 (d, J=9.9 Hz, 1H), 7.65 (d, J=7.5 Hz, 1H), 7.43 (dd, J=7.5 Hz, J=7.5 Hz, 1H), 7.31-7.25 (m, 1H), 7.18 (s, 1H), 6.15 (s, 1H), 5.88-5.75 (m, 1H), 5.48 (d, J=8.7 Hz, 1H), 5.25 (d, J=17.4 Hz, 1H), 5.13 (d, J=9.6 Hz, 1H), 4.73 (s, 1H), 4.68 (d, J=12.6 Hz, 1H), 4.54 (dd, J=9.0 Hz, J=8.3 Hz, 1H), 4.24-4.18 (m, 2H), 2.92-2.86 (m, 1H), 2.76-2.61 (m, 2H), 2.19-1.97 (m, 4H), 1.87-1.23 (m, 11H), 1.18-0.84 (m, 8H).

Compound I-12 was also prepared via another route shown in the following scheme:

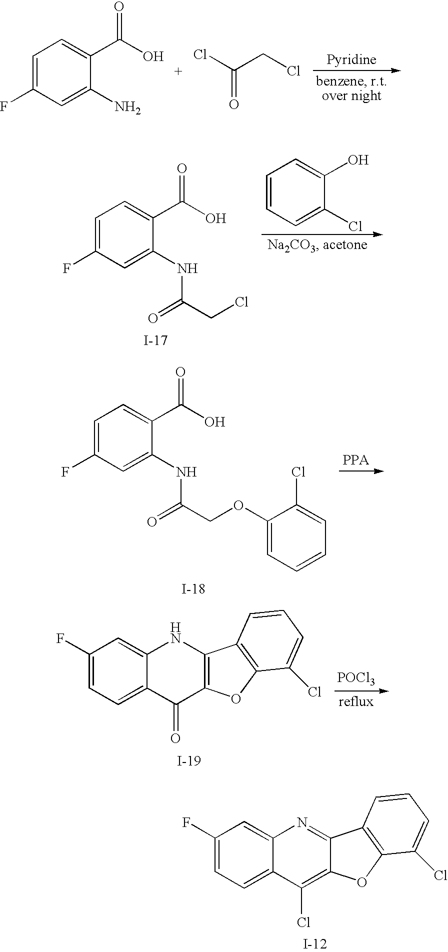

Pyridine (24.01 g, 303.0 mmol) was added dropwise to a solution of 2-amino-4-fluoro-benzoic acid (9.00 g, 58.0 mmol) in benzene (300 mL). The resulting solution was stirred for 10 min at room temperature, followed by slow addition of chloroacetyl chloride (12 mL, 151. 0 mmol). Stirring was continued for additional 12 hours. After removal of the solvent under vacuum, the residue was dissolved in EA (400 mL) and washed with 6N HCl. The organic layer was washed with brine, dried over MgSO₄, and evaporated under vacuum to give a crude product, which was recrystallized from ethyl acetate and n-hexan to afford compound I-17 (8.81 g, 65%). MS: m/z 254.0, 256.0 (M⁺+1); ¹H NMR (CD₃OD) δ: 8.45 (dd, J=12.0 Hz, J=2.7 Hz, 1H), 8.16 (dd, J=12.0 Hz, J=6.6 Hz, 1H), 6.95-6.88 (m, 1H), 4.31 (s, 2H).

A mixture of compound I-17 (5.00 g, 21.6 mmol), 2-chlorophenol (8.35 g, 64.8 mmol) and K₂CO₃ (10.0 g, excess) in dry THF (100 mL) was heated at 85° C for 12 hours. After cooled to room temperature, the reaction mixture was filtered. The obtained solid was washed with 1N HCl, brine, dried over MgSO₄, evaporated under vacuum, and recrystallized from CH₂Cl₂ and n-hexan to afford compound I-18 as a powder (3.41 g, 10.5 mmol, 49%). MS: m/z 346.0, 348.0 (M⁺+1); ¹H NMR (DMSO-d⁶) δ: 8.44 (dd, J=12.0 Hz, J=2.4 Hz, 1H), 8.04 (dd, J=12.0 Hz, J=6.9 Hz, 1H), 7.44 (dd, J=7.8 Hz, J=1.2 Hz, 1H), 7.31-7.24 (m, 1H), 7.15-7.12 (m, 1H), 7.04-6.97 (m, 2H), 4.83 (s, 2H).

Compound I-17 (3.00 g, 9.3 mmol) in polyphosphoric acid (20 g) was heated and stirred at 120-130 ° C. for 8 hours. After cooled to room temperature, the reaction mixture was treated with crushed ice, basified with sodium carbonate. The resulting suspension was filtered, washed with water, and dried to give compound I-19(1.36 g, 51%). MS m/z 288.0, 290.0 (M⁺+1).

A solution of compound I-19 (3.00 g, 10.4 mmol) in excess phosphorus oxychloride (POCl₃) was refluxed for 4 hours. The solution was cooled and thoroughly concentrated. The residue was subjected to extraction with methylene chloride and 10% sodium hydroxide. The organic layer was dried over MgSO₄, concentrated, and recrystallized from CH₂Cl₂ and n-hexane to afford compound I-12 (2.06 g, 65%). MS m/z 306.0, 308.0 (M⁺+1).

### EXAMPLES 14-44

### Syntheses of Compounds 14-44

Compounds 14-38 were synthesized in manners similar to that described in Example 13.

Compound 14: MS m/z 790.3 (M⁺+1).

Compound 15: MS m/z 790.3 (M⁺+1) ; ¹H NMR (CDCl₃) δ 8.27 (d, J=7.5 Hz, 1H), 8.13 (dd, J=9.3 Hz, J=6.0 Hz, 1H), 8.07 (s, 1H), 7.71 (d, J=10.8 Hz, 1H), 7.63-7.39 (m, 3H), 7.17-7.09 (m, 1H), 6.10 (s, 1H), 5.83-5.68 (m, 1H), 5.66 (d, J=9.0 Hz, 1H), 5.18 (d, J=17.4 Hz, 1H), 5.06 (d, J=10.5 Hz, 1H), 4.71 (s, 1H), 4.63-4.57 (m, 2H), 4.16-4.08 (m, 2H), 2.88-2.79 (m, 1H), 2.72-2.47 (m, 2H), 2.19-1.84 (m, 9H), 1.73-0.84 (m, 13H).

Compound 16: MS m/z 820.2 (M⁺+1).

Compound 17: MS m/z 804.3 (M⁺+1); ¹H NMR (CDCl₃) δ 10.13 (s, 1H), 8.17 (dd, J=9.3 Hz, J=6.0 Hz, 1H), 7.73 (d, J=10.5 Hz, 1H), 7.43 (s, 2H), 7.35 (s, 1H), 7.19 (m, 1H), 6.15 (s, 1H), 5.88-5.70 (m, 1H), 5.55 (d, J=9.0 Hz, 1H), 5.20 (d, J=16.8 Hz, 1H), 5.08 (d, J=10.5 Hz, 1H), 4.70 (brs, 1H), 4.62-4.51 (m, 2H), 4.15-4.08 (m, 2H), 2.92-2.83 (m, 1H), 2.68-2.43 (m, 4H), 2.51 (s, 3H), 2.17-1.92 (m, 3H), 1.76-0.82 (m, 18H).

Compound 18: MS m/z 820.3 (M⁺+1); ¹H NMR (CDCl₃) δ 10.18 (s, 1H), 8.22 (dd, J=8.4 Hz, J=6.3 Hz, 1H), 7.78 (d, J=9.6 Hz, 1H), 7.38 (dd, J=7.8 Hz, J=7.5 Hz, 1H), 7.26-7.19 (m, 1H), 7.15 (d, J=7.8 Hz, 1H), 6.21 (s, 1H), 5.84-5.71 (m, 1H), 5.49 (d, J=9.0 Hz, 1H), 5.21 (d, J=17.1 Hz, 1H), 5.10 (d, J=10.5 Hz, 1H), 4.74 (brs, 1H), 4.62 (d, J=12.0 Hz, 1H), 4.58-4.48 (m, 1H), 7.19-4.12 (m, 2H), 4.04 (s, 3H), 2.90-2.85 (m, 1H), 2.72-2.51 (m, 2H), 2.15-1.93 (m, 6H), 1.76-0.82 (m, 18H).

Compound 19: MS m/z790.3 (M⁺+1); ¹H NMR (CDC1₃) δ 10.13 (s, 1H), 8.31 (d, J=7.8 Hz, 1H), 8.14 (dd, J=8.6 Hz, J=5.1 Hz, 1H), 7.84 (d, J=9.6 Hz, 1H), 7.67-7.54 (m, 2H), 7.49-7.38 (m, 2H), 7.28 (s, 1H), 6.18 (s, 1H), 5.83-5.70 (m, 1H), 5.54 (d, J=8.7 Hz, 1H), 5.21 (d, J=17.4 Hz, 1H), 5.09 (d, J=10.2 Hz, 1H), 4.77 (s, 1H), 4.69 (d, J=12.0 Hz, 1H), 4.54 (dd, J=9.5 Hz, J=8.7 Hz, 1H), 4.16-4.06 (m, 2H), 2.90-2.86 (m, 1H), 2.66-2.56 (m, 2H), 2.13-1.93 (m, 6H), 1.71-0.74 (m, 16H).

Compound 20: MS m/z 808.2 (M⁺+1); ¹H NMR (CDCl₃) δ 10.09 (s, 1H), 8.29 (d, J=7.5 Hz, 1H), 7.99 (dd, J=12.0 Hz, J=8.7 Hz, 1H), 7.89 (dd, J=12.0 Hz, 8.1 Hz, 1H), 7.68-7.55 (m, 2H), 7.46 (dd, J=7.5 Hz, J=7.2 Hz, 1H), 7.37 (s, 1H), 6.18 (s, 1H), 5.84-5.69 (m, 1H), 5.56 (d, J=9.3 Hz, 1H), 5.21 (d, J=17.4 Hz, 1H), 5.10 (d, J=10.5 Hz, 1H), 4.78 (brs, 1H), 4.69 (d, J=12.0 Hz, 1H), 4.55 (dd. J=8.4 Hz, J=7.5 Hz, 1H), 4.14-4.03 (m, 2H), 2.92-2.81 (m, 1H), 2.69-2.48 (m, 2H), 2.19-2.02 (m, 2H), 2.01-1.81 (m, 4H), 1.78-1.07 (m, 8H), 1.01-0.88 (8H).

Compound 21: MS m/z 790.2 (M⁺+1); ¹H NMR (CDCl₃) δ 10.27 (s, 1H), 8.33 (d, J=7.5 Hz, 1H), 7.98 (d, J=8.4 Hz, 1H), 7.69-7.51 (m, 4H), 7.47 (dd, J=7.5 Hz, J=7.0 Hz, 1H), 7.16 (s, 1H), 7.09 (dd, J=12.6 Hz, J=7.5 Hz, 1H), 6.12 (s, 1H), 5.86-5.74 (m, 1H), 5.38 (d, J=9.0 Hz, 1H), 5.24 (d, J=17.1 Hz, 1H), 5.11 (d, J=10.2 Hz, 1H), 4.58-4.50 (m, 2H), 4.15-4.02 (m, 2H), 2.89-2.85 (m, 1H), 2.66-2.46 (m, 2H), 2.17-1.96 (m, 6H), 1.58-0.68 (m, 16H).

Compound 22: MS m/z 772.3 (M⁺+1); ¹H NMR (CDCl₃) δ 9.19 (brs, 1H), 8.33 (d, J=7.2 Hz, 1H), 8.17 (d, J=7.2 Hz, 1H), 8.15 (d, J=6.9 Hz, 1H), 7.68-7.39 (m, 6H), 6.15 (s, 1H), 5.84-5.69 (m, 2H), 5.20 (d, J=17.4 Hz, 1H), 5.09 (d, J=10.5 Hz, 1H), 4.73 (s, 1H), 4.66-4.55 (m, 2H), 4.21-4.06 (m, 2H), 2.92-2.81 (m, 1H), 2.69-2.44 (m, 2H), 2.18-2.04 (m, 2H), 1.98-1.90 (m, 1H), 1.74-1.34 (m, 8H), 1.31-1.12 (m, 3H), 1.04-0.88 (m, 8H).

Compound 23: MS m/z 832.3 (M⁺+1).

Compound 24: MS m/z 832.3 (M⁺+1); ¹H NMR (CDCl₃) δ 8.03 (d, J=9.0 Hz, 1H), 7.84 (d, 7.8 Hz, 1H), 7.62 (s, 1H), (s, 1H), 7.33 (dd, J=7.8 Hz, J=7.5 Hz, 1H), 7.07 (d, J=7.5 Hz, 1H), 7.06 (d, J=9.0 Hz, 1H), 6.16 (s, 1H), 5.86-5.73 (m, 1H), 5.61 (brs, 1H), 5.19 (d, J=16.8 Hz, 1H), 5.07 (d, J=9.9 Hz, 1H), 4.79 (s, 1H), 4.64-4.55 (m, 2H), 4.24-4.08 (m, 2H), 4.03 (s, 3H), 3.93 (s, 3H), 2.86 (brs, 1H), 2.69-248 (m, 2H), 2.18-1.92 (m, 3H), 1.76-1.10 (m, 12H), 1.05-0.82 (m, 8H).

Compound 25: MS m/z 836.2 (M⁺+1); ¹H NMR (CDCl₃) δ 10.22 (s, 1H), 8.30 (s, 1H), 8.09 (d, J=9.3 Hz, 1H), 7.59-7.48 (m, 3H), 7.16 (s, 1H), 7.12 (s, 1H), 6.16 (s, 1H), 5.86-5.73 (m, 1H), 5.47 (d, J=9.3 Hz, 1H), 5.22 (d, 16.8 Hz, 1H), 5.12 (d, J=10.2 Hz, 1H), 4.78 (brs, 1H), 4.63 (d, 12.3 Hz, 1H), 4.51 (dd, J=9.0 Hz, J=8.4 Hz, 1H), 4.21-4.08 (m, 2H), 3.97 (s, 3H), 2.93-2.60 (m, 1H), 2.68-2.58 (m, 2H), 2.18-1.97 (m, 3H), 1.67-0.82 (m, 19H).

Compound 26: MS m/z 830.3 (M⁺+1); ¹H NMR (CDCl₃) δ 8.05 (s, 1H), 7.91 (d, J=4.5 Hz, 1H), J=7.90 (d, J=9.0 Hz, 1H), 7.91 (s, 1H), 7.23 (s, 1H), 6.94 (d, J=9.0 Hz, 1H), 6.02 (s, 1H), 5.84-5.68 (m, 1H), 5.66 (d, J=9.0 Hz, 1H), 5.14 (d, J=17.1 Hz, 1H), 5.05 (d, J=10.5 Hz, 1H), 4.77 (s, 1H), 4.61 (dd, J=7.2 Hz, 9.4 Hz, 1H), 4.53 (d, J=12.0 Hz, 1H), 4.16 (dd, J=8.7 Hz, J=8.7 Hz, 1H), 4.06 (dd, J=8.7 Hz, J=4.2 Hz, 1H), 3.91 (s, 1H), 3.45 (q, J=6.9 Hz, 1H), 2.93-2.83 (m, 1H), 2.68-2.59 (m, 1H), 2.54-2.42 (m, 1H), 2.36 (s, 3H), 2.34 (s, 3H), 2.18-2.04 (m, 2H), 1.95 (dd, J=7.6 Hz, J=5.4 Hz, 1H), 1.74-1.13 (m, 12H), 1.03-0.88 (8H).

Compound 27: MS m/z 802.3 (M⁺+1).

Compound 28: MS m/z 802.3 (M⁺+1); ¹H NMR (CDCl₃) δ 8.30 (d, J=7.5 Hz, 1H), 8.06 (d, J=9.0 Hz, 1H), 7.69-7.53 (m, 2H), 7.47-4.41 (m, 2H), 7.09 (d, J=9.3 Hz, 1H), 6.16 (s, 1H), 5.83-5.71 (m, 1H), 5.48 (d, J=9.0 Hz, 1H), 5.20 (d, J=16.8 Hz, 1H), 5.08 (d, J=10.5 Hz, 1H), 4.71 (brs, 1H), 4.61 (d, J=12 Hz, 1H), 4.53 (dd, J=9.3 Hz, J=8.4 Hz, 1H), 4.17-4.09 (m, 2H), 3.93 (s, 3H), 3.74 (s, 1H), 2.92-2.83 (m, 1H), 2.72-2.43 (m, 2H), 2.41-1.91 (m, 8H), 1.74-0.84 (m, 14H)

Compound 29: MS m/z 832.3 (M⁺+1).

Compound 30: MS m/z 832.3 (M⁺+1); ¹H NMR (CDCl₃) δ 8.05 (d, J=9.3 Hz, 1H), 7.23 (s, 1H), 7.52 (s, 1H), 7.45 (s, 1H), 7.44 (d, J=9.3 Hz, 1H), 7.26 (s, 1H), 7.18 (d, J=9.0 Hz, 1H), 7.09 (d, J=9.0 Hz, 1H), 6.13 (s, 1H), 5.85-5.72 (m, 1H), 5.66 (d, J=8.7 Hz, 1H), 5.21 (d, J=17.4 Hz, 1H), 5.09 (d, J=10.2 Hz, 1H), 4.77 (s, 1H), 4.58 (dd, J=12.7 Hz, J=11.7 Hz, 1H), 4.21-4.08 (m, 2H), 3.94 (s, 3H), 3.92 (s, 3H), 2.91-2.82 (m, 1H), 2.68-2.47 (m, 2H), 2.18-1.92 (m, 3H), 1.76-1.16 (m, 7H), 1.04-0.82 (m, 12H).

Compound 31: MS m/z 830.3 (M⁺+1); ¹H NMR (CDCl₃) δ 8.18 (s, 1H), 7.98 (d, J=9.6 Hz, 1H), 7.51-7.33 (m, 2H), 7.02 (dd, J=9.2 Hz, J=2.1 Hz, 1H), 6.11 (s, 1H), 5.85-5.73 (m, 1H), 5.48 (d, J=8.7 Hz, 1H), 5.19 (d, J=16.8 Hz, 1H), 5.10 (d, J=10.8 Hz, 1H), 4.74 (s, 1H), 4.61-4.54 (m, 2H), 4.25-4.08 (m, 2H), 3.91 (s, 3H), 3.54-3.43 (m, 3H), 2.92-2.78 (m, 4H), 2.69-2.41 (s, 2H), 2.16-1.96 (m, 3H), 1.61-0.88 (m, 20H).

Compound 32: MS m/z 816.3 (M⁺+1); ¹H NMR (CDCl₃) δ 8.04-7.92 (m, 3H), 7.42-7.32 (m, 3H), 7.49 (d, J=9.0 Hz, 1H), 6.05 (s, 1H), 5.83-5.64 (m, 2H), 5.16 (d, J=17.4 Hz, 1H), 5.63 (d, J=10.5 Hz, 1H), 4.75 (s, 1H), 4.58 (dd, J=12.6 Hz, J=10.8 Hz, 2H), 4.22-4.06 (m, 2H), 3.92 (s, 3H), 2.92-2.81 (m, 1H), 2.70-2.56 (m, 1H), 2.47 (s, 3H), 2.19-2.04 (m, 2H), 1.98-1.91 (m, 1H), 1.72-1.14 (m, 12H), 1.04-0.89 (m, 8H).

Compound 33: MS m/z 832.3 (M⁺+1); ¹H NMR (CDCl₃) δ 8.12 (d, J=8.4 Hz, 1H), 8.04 (d, J=9.3 Hz, 1H), 7.42 (s, 1H), 7.08-6.96 (m, 3H), 6.06 (s, 1H), 5.74-5.70 (m, 1H), 5.67 (brs, 1H), 5.19 (d, 16.5 Hz, 1H), 5.09 (d, J=9.6 Hz, 1H), 4.81 (s, 1H), 4.65-4.52 (m, 2H), 4.26-4.04 (m, 3H), 3.92 (s, 6H), 2.94-2.79 (m, 1H), 2.71-2.46 (m, 2H), 2.16-1.93 (m, 3H), 1.74-1.17 (m, 11H), 1.04-0.84 (m, 8H).

Compound 34: MS m/z 846.3 (M⁺+1); ¹H NMR (CDCl₃) δ 8.12 (d, J=8.7 Hz, 1H), 7.99 (d, J=9.6 Hz, 1H), 7.44 (s, 1H), 7.06-6.96 (m, 3H), 6.08 (s, 1H), 5.85-5.71 (m, 1H), 5.53 (d, J=9.0 Hz, 1H), 5.20 (d, J=17.4 Hz, 1H), 5.10 (d, J=10.5 Hz, 1H), 4.84 (s, 1H), 4.63-4.51 (m, 2H), 4.25-4.04 (m, 5H), 3.94 (s, 3H), 2.96-2.85 (m, 1H), 2.69-2.46 (m, 2H), 2.20-1.21 (m, 17H), 1.06-0.82 (m, 8H).

Compound 35: MS m/z 820.3 (M⁺+1); ¹H NMR (CDCl₃) δ 10.18 (s, 1H), 8.09 (d, J=9.0 Hz, 1H), 7.98 (d, J=7.8 Hz, 1H), 7.52 (dd. J=9.3 Hz, J=3.6 Hz, 1H), 7.48 (s, 1H), 7.33 (dd, J=7.8 Hz, J=7.8 Hz, 1H), 7.13 (d, J=9.0 Hz, 1H), 6.16 (s, 1H), 5.86-5.73 (m, 1H), 5.55 (d, J=9.0 Hz, 1H), 5.22 (d, J=16.8 Hz, 1H), 5.11 (d, J=10.8 Hz, 1H), 4.77 (s, 1H), 4.63 (d, J=10.4 Hz, 1H), 4.53 (dd, J=9.0 Hz, J=9.0 Hz, 1H), 4.22-4.06 (m, 2H), 3.96 (s, 3H), 2.96-2.84 (m, 1H), 2.68-2.50 (m, 2H), 2.18-2.05 (m, 2H), 2.02-1.94 (m, 1H), 1.84-1.21 (m, 12H), 1.04-0.82 (m, 8H).

Compound 36: MS m/z 802.3 (M⁺+1); ¹H NMR (CDCl₃) δ 8.36 (d, J=7.5 Hz, 1H), 7.74 (d, J=8.7 Hz, 1H), 7.58 (d, J=8.7 Hz, 1H), 7.47-7.33 (m, 3H), 7.03 (d, J=6.9 Hz, 1H), 6.17 (s, 1H), 5.84-5.73 (m, 1H), 4.45 (d, J=9.0 Hz, 1H), 5.16 (d, J=17.4 Hz, 1H), 5.08 (d, J=9.9 Hz, 1H), 4.95-4.86 (m, 1H), 4.66-4.55 (m, 2H), 4.28-4.20 (m, 1H), 4.13 (s, 3H), 2.96-2.87 (m, 1H), 2.78-2.54 (m, 4H), 2.18-1.24 (m, 14H), 1.08-0.92 (8H).

Compound 37: MS m/z 840.3 (M⁺+1); ¹H NMR (CDCl₃) δ 10.11 (s, 1H), 8.48 (s, 1H), 8.35 (d, J=7.5 Hz, 2H), 7.71-7.58 (m, 3H), 7.50 (dd, J=7.5 Hz, J=7.2 Hz, 1H), 6.23 (brs, 1H), 5.84-5.71 (m, 1H), 5.53 (d, J=8.7 Hz, 1H), 5.22 (d, J=17.4 Hz, 1H), 5.10 (d, J=10.5 Hz, 1H), 4.69 (d, 12.0 Hz, 1H), 4.61-4.53 (m, 2H), 4.16-4.04 (m, 2H), 2.92-2.83 (m, 1H), 2.73-2.51 (m, 2H), 2.18-1.90 (m, 5H), 1.74-0.77 (m, 18H).

Compound 38: MS m/z 808.2 (M⁺+1).

Compound 39: MS m/z 834.5 (M⁺+1); ¹H NMR (CDCl₃) δ 8.01 (d, J=8.7 Hz, 1H), 7.70 (s, 1H), 7.46-7.36 (m, 2H), 7.17 (d, J=8.7 Hz, 1H), 7.03 (d, J=8.4 Hz, 1H), 6.07 (s, 1H), 5.82-5.64 (m, 1H), 5.17 (d, J=17.1 Hz, 1H), 5.05 (d, J=9.9 Hz, 1H), 4.68-4.50 (m, 2H), 4.29 (d, J=8.7 Hz, 1H), 4.13-4.04 (m, 1H), 3.92 (s, 6H), 2.86-2.76 (m, 1H), 2.63-2.73 (m, 1H), 2.58-2.44 (m, 1H), 2.32-1.88 (m, 2H), 1.40-0.84 (m, 6H), 1.38 (s, 9H), 1.05 (s, 9H).

Compound 40: MS m/z 791.3 (M⁺+1).

Compound 41: MS m/z 850.3 (M⁺+1).

Compound 42: MS m/z 807.3 (M⁺+1).

Compound 43: MS m/z 807.3 (M⁺+1); ¹H NMR (CDCl₃) δ 8.20 (dd, J=9.3 Hz, J=6.0 Hz, 1H), 7.98 (s, 1H), 7.90 (dd, J=7.5 Hz, J=0.9 Hz, 1H), 7.77 (dd, J=10.4 Hz, J=2.7 Hz, 1H), 7.40-7.26 (m, 2H), 7.21-7.13 (m, 2H), 6.24 (brs, 1H), 5.89-5.76 (m, 1H), 5.37 (d, J=8.7 Hz, 1H), 5.20 (d, J=16.5 Hz, 1H), 5.09 (d, J=12.0 Hz, 1H), 4.58-4.43 (m, 3H), 4.22-4.16 (dd, J=12.2 Hz, J=3.6 Hz, 1H), 4.04 (s, 3H), 2.61-2.56 (m, 1H), 2.18-1.16 (m, 19H), 1.01 (d, J=5.1 Hz, 3H), 0.90 (d, J=6.6 Hz, 3H).

Compound 44: MS m/z 827.3 (M⁺+1).

### EXAMPLE 45

### Inhibition of NS3/4A Protein

### Protein Expression and Purification

A plasmid containing N-terminal His₆-tagged-NS4A₍₂₁₋₃₂₎-GSGS-NS3(₃₋₁₈₁₎ was transformed into E. coli strain BL21(DE3)pLysS (Novagen) for protein over-expression. Single colony of transformed BL21 (DE3)pLysS was cultured in 200 mL of Lauria-Bertani (LB) medium with Kanamycin and Chloramphenicol at 37° C. overnight. The bacterial culture was transferred into 6 L LB medium (Difco) containing antibiotics and incubated with shaking at 22° C. After the absorbance at 600 nm reached 0.6, the culture was induced with 1 mM isopropyl-1-thio-β-D-galactopyranoside (IPTG) at 22° C. for 5 hours. The culture was subsequently harvested by centrifugation (6,000× g for 15 minutes at 4° C.). Cell pellets were resuspended in 150 mL buffer A (50 mM HEPES, pH 7.4, 0.3 M NaCl, 0.1% (w/v) CHAPS, 10 mM imidazol, 10% (v/v) glycerol). After four passes through a Microfluidizer operated at 30 psi disrupted the mixture, the cell debris was removed by centrifugation (58,250× g for 30 minutes at 4° C.). The cell lysate containing His₆-tagged proteins was applied at 3 mL/min to a 25 ml Ni-NTA (Qiagen) column in the presence of 10 mM imidazole using a GradiFrac system (Pharmacia). The column was washed with 10 column volumes of the lysis buffer. The bound NS4A₍₂₁₋₃₂₎-GSGS-NS3₍₃₋₁₈₁₎ was eluted with 8 column volumes of buffer A supplemented with 300 mM imidazole. The pooled fractions were further purified by Q-Sepharose column equilibrated in buffer B (50 mM HEPES, pH 7.4, 0.1% (w/v) CHAPS, 10% (v/v) glycerol, 5 mM dithiothreitol (DTT), and 1 M NaCl). The eluant containing NS4A₍₂₁₋₃₂₎-GSGS-NS3₍₃₋₁₈₁₎ was collected. Fractions containing NS4A₍₂₁₋₃₂₎-GSGS-NS3₍₃₋₁₈₁₎ we collected and further purified by size-exclusion chromatography using Sephacryl-75 columns (16×100 cm, Pharmacia) at a flow rate of 0.5 mL/min. Columns were pre-equilibrated in buffer C (50 HEPES, pH 7.4, 0.1% (w/v) CHAPS, 5 mM DTT, 10% (v/v) glycerol). The purified protein was frozen and stored at −80° C. before use.

### Inhibition Assay Protocol

The HPLC Microbore assay for separation of HCV protease substrate and products was used. The substrate used in the assay was Ac-Asp-Glu-Asp(EDANS)-Glu-Glu-Abu-ψ-[COOAla]-Ser-Lys(DABCYL)-NH₂ (RET S1, ANASPEC). The buffer used in the assay included 50 mM Tris buffer, pH 7.4, 100 mM NaCl, 20% glycerol, and 0.012% CHAPS.

A stock aqueous solution of 10 mM substrate RET S1 was prepared and stored in aliquots at −80° C. before use. DTT, RET S 1, and a test compound were dissolved in the buffer (the final volume: 80 μL), which was added to a well of a 96-well plate. Reaction was initiated by addition of 20 μL of 10 nM NS3/4A protease in the buffer to form a 100 μL assay solution, which contained 50 mM Tris, pH 7.4, 100 mM NaCl, 20% glycerol, 0.012% CHAPS, 10 mM DTT, 5 μM substrate RET S1, and 10 μM the test compound. The final concentration of NS3/4A protease was 2 nM, which was lower than the Km of substrate RET S1.

The assay solution was incubated for 30 minutes at 30° C. The reaction was then terminated by addition of 100 μL of 1% TFA. 200 μL aliquot was transferred to each well of Agilent 96-well plates for the next step.

Separation of Product from Substrate

The reaction products were analyzed using reverse phase HPLC described below. The HPLC system consisted of: Agilent 1100, Degasser G1379A, Binary pump G1312A, Autosampler G1367A, Column thermostated chamber G1316A, Diode array detector G1315B, Column: Agilent, ZORBAX Eclipse XDB-C18, 4.6 mm, 5 μm, P/N 993967-902, Column thermostat: room temperature, Injection volume: 100 μL; Solvent A=HPLC grade water+0.09% TFA, Solvent B=HPLC grade acetonitrile+0.09% TFA. Total HPLC running time was 7.6 minutes with a linear gradient of acetonitrile from 25 to 50% B within 4 minutes, 50% B for 30 seconds, and a gradient from 50 to 25% B within 30 seconds. The column was re-equilibrated with 25% B for 2.6 minutes before the next sample was injected. The IC₅₀ value (the concentration at which 50% inhibition of NS3/4A was achieved) was calculated for each test compound based on the HPLC results.

Compounds 1-44 were tested in this assay. The results showed that almost all test compounds exhibited inhibition of NS3/4A protease activity. Some compounds surprisingly had very low IC₅₀ values. For example, 36 compounds had IC₅₀ values lower than 50 nM and 5 compounds had IC₅₀ values between 50-500 nM.

### EXAMPLE 46

### HCV Replicon Cell Assay Protocol

HCV replicon Cells were maintained in DMEM containing 10% fetal bovine serum (FBS), 1.0 mg/ml G418, and appropriate supplements (media A).

On day 1, the replicon cell monolayer was treated with a trypsin/EDTA mixture, removed, and diluted with media A to give a final concentration of 48,000 cells/ml. The solution (1 ml) was plated into each well of a 24-well tissue culture plate, and cultured overnight in a tissue culture incubator at 37° C. with 5% CO₂.

On day 2, each test compound (in DMSO) was diluted with DMEM containing 10% FBS and appropriate supplements to provide a series of sample solutions having different concentrations. The final concentration of DMSO was maintained at 0.2% throughout the dilution series.

Media of the replicon cell monolayer was removed, and then the sample solutions were added. DMEM containing 10% FBS and appropriate supplements but no compound was added to other wells as no-compound controls.

The cells were incubated with a compound or 0.2% DMSO in a media the same as media A described above except G418 is absent for 72 hours in a tissue culture incubator with 5% CO₂ at 37° C. The media was removed, and the replicon cell monolayer was washed once with PBS and extracted total cellular RNA. RNA extraction reagents (such as reagents from RNeasy kits or TRIZOL reagents) were added to the cells immediately to avoid degradation of RNA. Total RNA was extracted according the manufacturer's instruction with modification to improve extraction efficiency and consistency. Finally, total cellular RNA, including HCV replicon RNA, was eluted and stored at −80° C. until further processing.

A TaqMan® real-time RT-PCR quantification assay was set up with two sets of specific primers and probe. One was for HCV and the other was for ACTB (beta-actin). The total RNA extractants from the treated HCV replicon cells were added to the PCR reactions for quantification of both HCV and ACTB RNA in the same PCR well. Experimental failure was flagged and rejected based on the level of ACTB RNA in each well. The level of HCV RNA in each well was calculated according to a standard curve run in the same PCR plate. The percentage of inhibition of HCV RNA level by the compound treatment was calculated using the DMSO or no-compound control as 0% of inhibition. EC₅₀ (the concentration at which 50% inhibition of HCV RNA level was achieved) was calculated from the titration curve of any given compound.

Compounds 1-44 were tested in the HCV replicon cell assay. The results showed that all test compounds exhibited inhibitory effect against the HCV RNA level. Some test compounds surprisingly had very low EC₅₀ values. For example, 33 compounds had EC₅₀ values lower than 50 nM and 1 compound had an EC₅₀ value between 50-500 nM.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula (I): 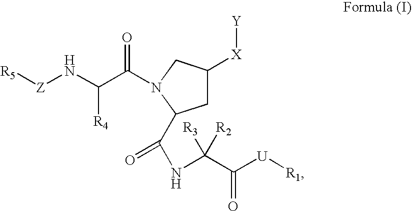
wherein
each of R₁, R₂, R₃ R4, and R₅, independently, is H, C₁₋₆ alkyl, C₁₋₆ alkoxyl, C₃₋₁₀ cycloalkyl, C₁₋₁₀ heterocycloalkyl, C₆₋₁₀ aryl, or C₃₋₁₀ heteroaryl; or R₂ and R₃, together with the carbon atom to which they are attached, form a C₃₋₁₀ cycloalkyl and C₁₋₁₀ heterocycloalkyl optionally having one or more substituents selected from a group consisting of halo, nitro, cyano, C₁₋₆ alkyl, C₁₋₆ alkoxyl, C₂₋₆ alkenyl, C₂₋₆ alkynyl, C₆₋₁₀ aryl, and C₃₋₁₀ heteroaryl;
U is —O—, —NH—, —C(O)NH—, —NHSO—, or —NHSO₂—;
X is —O—, —S—, —NH—, or —OCH₂—;
Y is 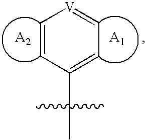
in which V is —CH—or —N—; and each of A₁ and A₂, independently, is selected from the group consisting of C₃₋₁₀ cycloalkyl, C₁₋₁₀ heterocycloalkyl, C₆₋₁₀ aryl, and C₃₋₁₀ heteroaryl, each of which is optionally substituted with halo, nitro, cyano, C₁₋₆ alkyl, C₁₋₆ alkoxyl, C₂₋₆ alkenyl, C₂₋₆ alkynyl, C₆₋₁₀ aryl, or C₃₋₁₀ heteroaryl, or optionally fused with another C₃₋₁₀ cycloalkyl, C₁₋₁₀ heterocycloalkyl, C₆₋₁₀ aryl, and C₃₋₁₀ heteroaryl, optionally substituted with halo, nitro, cyano, C₁₋₆ alkyl, C₁₋₆ alkoxyl, C₂₋₆ alkenyl, C₂₋₆ alkynyl, C₆₋₁₀ aryl, or C₃₋₁₀ heteroaryl; and
Z is —C(O), —O—C(O)—, —NH—C(O)—, —O—C(S)—, —NH—C(S)—, —O—C(NH)—, or —NH—C(NH)—.

2. The compound of claim 1, wherein R₂ and R₃, together with the carbon atom to which they are attached, form cyclopropyl.

3. The compound of claim 2, wherein the cyclopropyl is substituted with vinyl.

4. The compound of claim 3, wherein Ar₂ is phenyl and V is —N—.

5. The compound of claim 4, wherein Y is selected from 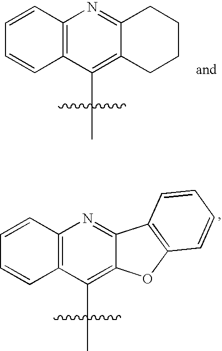
each of which is optionally substituted with halo, C₁₋₆ alkyl, or C₁₋₆ alkoxyl.

6. The compound of claim 5, wherein X is —O—.

7. The compound of claim 6, wherein U is —NHSO₂—and Z is —OC(O)—.

8. The compound of claim 7, wherein R₁ is cyclopropyl, R₄ is C₁₋₆ alkyl, and R₅ is cyclopentyl.

9. The compound of claim 1, wherein X is —O—.

10. The compound of claim 1, wherein Ar₂ is phenyl and V is —N—.

11. The compound of claim 1, wherein Y is 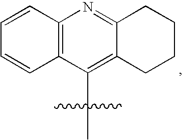
which is optionally substituted with halo, C₁₋₆ alkyl, or C₁₋₆ alkoxyl.

12. The compound of claim 1, wherein Y is 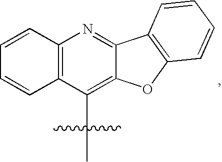
which is optionally substituted with halo, C₁₋₆ alkyl, or C₁₋₆ alkoxyl.

13. The compound of claim 1, wherein U is —NHSO₂—and R₁ is cyclopropyl.

14. The compound of claim 1, wherein Z is —OC(O)— and R₅ is cyclopentyl.

15. The compound of claim 1, wherein R₁ is cyclopropyl and R4 is C₁₋₆ alkyl.

16. The compound of claim 1, wherein Y is selected from 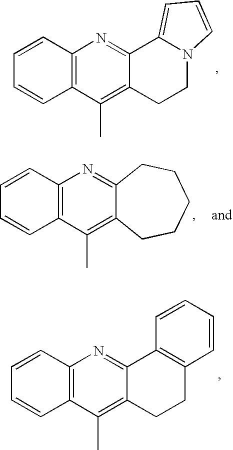
each of which is optionally substituted with halo, C₁₋₆ alkyl, or C₁₋₆ alkoxyl.

17. The compound of claim 1, wherein the compound have the stereochemistry as shown in the following formula: 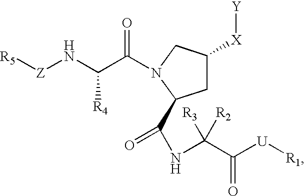
wherein R₁, R₂, R₃, R4, R₅, U, X, Y, and Z are as defined in claim 1.

18. The compound of claim 1, wherein the compound is one of compounds 1-44.

19. A method for treating hepatitis C virus infection, comprising administering to a subject in need thereof an effective amount of a compound of claim 1.

20. The method of claim 1, wherein the compound is one of compounds 1-44.

21. The method of claim 19, further comprising administering to the subject an effective amount of an immunomodulatory agent.

22. The method of claim 19, further comprising administering to the subject an effective amount of another antiviral agent.

23. The method of claim 19, further comprising administering to the subject an effective amount of another inhibitor of HCV protease.

24. The method of claim 19, further comprising administering to the subject an effective amount of an inhibitor of a target in the HCV life cycle other than HCV NS3 protease.

25. A pharmaceutical composition, comprising a compound of claim 1 and a pharmaceutically acceptable carrier.
